(19) United States
(12) Patent Application Publication
(10) Pub. No.: US 2014/0142071 A1
(43) Pub. Date: May 22, 2014

(54) REGRESSION OF ARTERIAL PLAQUE

(71) Applicant: ATHERONOVA OPERATIONS, INC., Irvine, CA (US)

(72) Inventors: Filiberto Zadini, Camarillo, CA (US); Giorgio Zadini, Camarillo, CA (US)

(73) Assignee: ATHERONOVA OPERATIONS, INC., Irvine, CA (US)

(21) Appl. No.: 14/164,648

(22) Filed: Jan. 27, 2014

Related U.S. Application Data
(63) Continuation of application No. 13/871,904, filed on Apr. 26, 2013.
Application No. 13/871,904 is a continuation of application No. 12/211,754, filed on Sep. 16, 2008, now abandoned.
Application No. 12/211,754 is a continuation-in-part of application No. 12/024,908, filed on Feb. 1, 2008, now Pat. No. 8,304,383.
Application No. 12/024,908 is a continuation-in-part of application No. 11/649,062, filed on Jan. 3, 2007, now abandoned.
Application No. 11/649,062 is a continuation-in-part of application No. 11/384,150, filed on Mar. 17, 2006, now abandoned.
Application No. 11/384,150 is a continuation-in-part of application No. 11/373,943, filed on Mar. 13, 2006, now abandoned.
Application No. 12/211,754 is a continuation-in-part of application No. 11/542,694, filed on Oct. 4, 2006, now abandoned.
Application No. 12/211,754 is a continuation-in-part of application No. PCT/US2006/044619, filed on Nov. 16, 2006.
Application No. PCT/US2006/044619 is a continuation of application No. 11/384,150, filed on Mar. 17, 2006, now abandoned.
Application No. 11/384,150 is a continuation of application No. 11/373,943, filed on Mar. 13, 2006, now abandoned.
Application No. 12/211,754 is a continuation-in-part of application No. PCT/US2007/001214, filed on Jan. 16, 2007.
(60) Provisional application No. 60/739,143, filed on Nov. 22, 2005.
(60) Provisional application No. 60/793,379, filed on Apr. 19, 2006.
(60) Provisional application No. 60/739,143, filed on Nov. 22, 2005.
(60) Provisional application No. 60/760,471, filed on Jan. 20, 2006.

(51) Int. Cl. A61K 31/575; A61K 31/4418; A61K 31/505; A61K 31/366; A61K 31/47; A61K 31/22; A61K 31/40; A61K 31/405

(52) U.S. Cl. CPC A61K 31/575; A61K 31/40; A61K 31/4418; A61K 31/405; A61K 31/366; A61K 31/47; A61K 31/22; A61K 31/505
USPC 514/170; 514/182; 514/171

(57) ABSTRACT

Some embodiments of the present invention provide pharmaceutical formulations, for treating atherosclerosis in a mammal, including a bile acid and/or a terpene atherosclerotic plaque emulsifier. Some embodiments provide methods for administering such pharmaceutical formulations. In some embodiments, pharmaceutical formulations include a combination of a bile acid and a terpene in amounts effective to result in plaque regression, and the amount of each individual emulsifier in the combination can be lower than an amount that is effective to result in plaque regression when the emulsifier is administered alone. In some embodiments, a statin can be administered simultaneously or sequentially with the pharmaceutical formulation.

### RELATED APPLICATIONS

This application is a continuation of U.S. application Ser. No. 13/871,904, filed on Apr. 26, 2013, which is a continuation of U.S. application Ser. No. 12/211,754, filed on Sep. 16, 2008, entitled “Dissolution of Arterial Plaque,” which is a continuation-in-part of U.S. application Ser. No. 12/024,908, filed on Feb. 1, 2008, entitled, “Dissolution of Arterial Plaque,” now U.S. Pat. No. 8,304,383, which is a continuation-in-part of U.S. application Ser. No. 11/649,062, filed Jan. 3, 2007, entitled “Dissolution of Arterial Cholesterol Plaques by Pharmacological Preparation,” which is a continuation-in-part of U.S. application Ser. No. 11/384,150, filed Mar. 17, 2006, entitled “Dissolution of Arterial Cholesterol Plaques by Pharmacological Preparation,” which is a continuation-in-part of U.S. application Ser. No. 11/373,943, filed Mar. 13, 2006, entitled “Dissolution of Arterial Cholesterol Plaques by Pharmacological Preparation,” which claims priority to U.S. Provisional Application No. 60/739,143, filed Nov. 22, 2005, entitled “Dissolution of Arterial Cholesterol Plaques by Pharmacological Preparation”; U.S. application Ser. No. 12/211,754 is also a continuation-in-part of U.S. application Ser. No. 11/542,694, filed Oct. 4, 2006, entitled “Dissolution of Arterial Cholesterol Plaques by Phytochemical Emulsifiers,” which claims priority to U.S. Provisional Application No. 60/793,379, filed Apr. 19, 2006, entitled “Dissolution of Arterial Cholesterol Plaques by Phytochemical Emulsifiers”; U.S. application Ser. No. 12/211,754 also claims priority to U.S. Provisional Application No. 60/930,410, filed May 15, 2007, entitled “Dissolution of Arterial Cholesterol Plaques by Pharmacologically Induced Elevation of Endogenous Biliary Salts”; U.S. application Ser. No. 12/211,754 is also a continuation-in-part of International Application No. PCT/US2006/044619, filed Nov. 16, 2006, entitled “Dissolution of Arterial Cholesterol Plaques by a Class of Pharmacological Compounds,” which claims priority to U.S. patent application Ser. No. 11/384,150, filed Mar. 17, 2006, U.S. patent application Ser. No. 11/373,943, filed Mar. 13, 2006, and U.S. Provisional Application No. 60/739,143, filed Nov. 22, 2005; U.S. application Ser. No. 12/211,754 is also a continuation-in-part of International Application No. PCT/US2007/001214, filed Jan. 16, 2007, entitled “Drug-Eluting Stent with Atherosclerotic Plaques Dissolving Pharmacological Preparation,” which claims priority to U.S. Provisional Application No. 60/760,471, filed Jan. 20, 2006, entitled “Drug-Eluting Stent with Atherosclerotic Plaque Dissolving Pharmacological Preparation”; the contents of all of the foregoing are hereby incorporated by reference herein in their entireties.

### FIELD OF THE INVENTIONS

Some embodiments of the invention provide pharmaceutical formulations useful in atherosclerotic plaque treatments in mammals. Certain embodiments described herein comprise detergents, emulsifiers, for example, bile acids, terpenes, and saponins, effective to emulsify and dissolve atherosclerotic plaque components, either in a plaque or in circulation, resulting in plaque regression and/or inhibition of atherogenesis.

### BACKGROUND OF THE INVENTIONS

Cardiovascular disease is a leading cause of death in the human population. This is especially true in developed countries, where an increasing incidence of obesity is considered a major contributing factor to cardiovascular and related diseases. For example, the incidence of heart disease as a cause of death was 12.4% in all World Health Organization States, whereas in the U.S., heart attacks account for nearly 30% of deaths. In addition, other disease states related to or exacerbated by impairment of cardiovascular function make cardiovascular disease the single greatest contributor to death and disability.

An underlying issue in cardiovascular disease is the development of atherosclerosis, a disease that affects vessels of the arterial circulation. Atherosclerosis is characterized by a chronic inflammatory response in the walls of blood vessels, in part due to deposition of lipoproteins, in particular low density lipoproteins (LDLs), which appears to be involved in, and likely the cause of, macrophage infiltration. Atherosclerosis is known to begin during childhood, with the rate of progression dependent on a variety of factors including diet, exercise, and genetic predisposition.

The earliest morphologically identifiable stage of plaque development is a fatty streak, an accumulation of macrophages that have ingested oxidized LDL, giving them an appearance of fat in vessel wall muscular tissue. Upon ingesting oxidized LDL, fatty streak macrophages accumulate numerous cytoplasmic vesicles, and are known as foam cells. In disease progression, the fatty streak develops into an established plaque, characterized by further accumulation of macrophages and an inflammatory infiltrate. Another important stage of plaque development involves foam cell death, in which the associated release of foam cell content further exacerbates the inflammatory reaction. In addition, cytokines released by damaged endothelial cells at the site of the developing plaque induce smooth muscle cell proliferation and migration to the vessel intima, resulting in the development of a fibrous cap that covers the plaque. Over time, calcification at the margins of the fibrous cap can occur.

Progressive growth and development of an atherosclerotic plaques results in a narrowing of the lumen of the afflicted vessel. Traditionally, narrowing of 75% or greater has been considered clinically significant. Recently, it has been discovered that, due to the inherent instability of many plaques, events such as heart attacks can occur, even when there is no sign of significant vessel narrowing.

Structurally unstable plaques can spontaneously rupture, releasing into the vessel lumen tissue fragments and plaque contents that initiate a clotting response. Although the resulting clot is effective to cover and stabilize the rupture, it intrudes into the vessel lumen, creating a stenotic region of reduced luminal diameter and obstructed blood flow. If the compromise to flow is significant, e.g., where the clot completely or nearly completely occludes the vessel lumen, ischemia can occur in downstream tissues. Where the vessel is a coronary artery or a cerebral artery, rupture-associated tissue ischemia can result in myocardial infarction or stroke, respectively. Significantly, the majority of fatal rupture events occur in vessel regions having little prior narrowing. But repeated, non-fatal plaque ruptures can also lead to stenosis and downstream tissue ischemia.

Because of the health risk posed by unstable plaques, there is now a recognized need for early plaque detection, such as soft, vulnerable plaques, prior to the patient becoming symptomatic. Early detection of vulnerable plaques can be especially useful to diagnose a need for a course of treatment designed to reduce the risk of a sudden ischemic event resulting from plaque rupture and/or designed to reduce the risk of ischemia resulting from the gradual development of stenotic regions in a vessel. Traditionally, treatment of stenosis in sensitive areas, such as the heart or the brain, has been accomplished by angioplasty techniques. Recently, maintaining patency of such vessels has become easier due to the advent of vascular stent devices.

In the past, detection and diagnosis of atherosclerosis has been difficult. For example, according to data in the U.S. from 2004, the first symptom of cardiovascular disease in over half of those diagnosed with atherosclerosis is heart attack or sudden death. Unfortunately, by the time obvious symptoms arise, the disease is usually quite advanced, and treatment options and clinical outcomes are limited. The recognition of contributing factors, such as the effect of cholesterol intake, obesity, and smoking, has led to an awareness of the benefit of preventative lifestyle choices in reducing the risk of developing atherosclerosis.

Advances have been made in both the diagnosis and treatment of cardiovascular disease. For example, 64 slice CT technology now makes it possible to evaluate the extent of cardiovascular disease through detection of calcifications in vessels. In addition, certain CT protocols make it possible to visualize early stage, vulnerable plaques. Such advances make it easier to detect atherosclerosis at early stages, providing an increased window of opportunity to treat the disease.

### SUMMARY OF THE INVENTIONS

While prior art treatments can effectively deal with some of the factors that contribute to the development of atherosclerotic plaques (e.g., use of statins to reduce cholesterol levels) or to open occluded vessels (e.g., angioplasty and vascular stents), there remains a need for treatments that effect regression of existing plaques and decrease plaque burden in patients.

Some embodiments of the present invention provide a pharmaceutical formulation, for treating atherosclerosis in a mammal, comprising a bile acid or a pharmaceutically acceptable salt, conjugate, hydrate, solvate, polymorph, or mixture thereof; and a terpene or a pharmaceutically acceptable salt, conjugate, hydrate, solvate, polymorph, or mixture thereof, in which the formulation is in an amount effective to result in an amount of emulsification of an atherosclerotic plaque in an artery of the mammal sufficient to result in regression of the plaque. As used herein, “regression” of an atherosclerotic plaque includes regression of a size of the plaque and/or a composition of a plaque, such as a fibrous cap, a lipidic component, or a cell type.

In some embodiments, a bile acid of the pharmaceutical formulation comprises hyodeoxycholic acid (HDCA), or a pharmaceutically acceptable salt, conjugate, hydrate, solvate, or polymorph thereof; deoxycholic acid (DCA), or a pharmaceutically acceptable salt, conjugate, hydrate, solvate, or polymorph thereof; or a mixture thereof.

In some embodiments, a terpene of the pharmaceutical formulation comprises a limonene, such as a D-limonene and/or an L-limonene, or a pharmaceutically acceptable salt, conjugate, hydrate, solvate, or polymorph thereof; perillic acid, such as S-perillic acid or D-perillic acid, or a pharmaceutically acceptable salt, conjugate, hydrate, solvate, or polymorph thereof; perillyl alcohol, such as S-perillyl alcohol or D-perillyl alcohol, or a pharmaceutically acceptable salt, conjugate, hydrate, solvate, or polymorph thereof; or a mixture thereof.

In some embodiments, a bile acid of the pharmaceutical formulation comprises HDCA or a pharmaceutically acceptable salt, conjugate, hydrate, solvate, polymorph, or mixture thereof, and a terpene of the pharmaceutical formulation comprises D-limonene or a pharmaceutically acceptable salt, conjugate, hydrate, solvate, polymorph, or mixture thereof.

In some embodiments, a bile acid of the pharmaceutical formulation comprises DCA or a pharmaceutically acceptable salt, conjugate, hydrate, solvate, polymorph, or mixture thereof, and a terpene of the pharmaceutical formulation comprises D-limonene or a pharmaceutically acceptable salt, conjugate, hydrate, solvate, polymorph, or mixture thereof.

In some embodiments, a bile acid of the pharmaceutical formulation comprises HDCA or a pharmaceutically acceptable salt, conjugate, hydrate, solvate, polymorph, or mixture thereof, and a terpene of the pharmaceutical formulation comprises S-perillic acid or a pharmaceutically acceptable salt, conjugate, hydrate, solvate, polymorph, or mixture thereof.

In some embodiments, a bile acid of the pharmaceutical formulation comprises DCA or a pharmaceutically acceptable salt, conjugate, hydrate, solvate, polymorph, or mixture thereof, and a terpene of the pharmaceutical formulation comprises S-perillic acid or a pharmaceutically acceptable salt, conjugate, hydrate, solvate, polymorph, or mixture thereof.

In some embodiments, a bile acid of the pharmaceutical formulation comprises HDCA or a pharmaceutically acceptable salt, conjugate, hydrate, solvate, polymorph, or mixture thereof, and a terpene of the pharmaceutical formulation comprises S-perillyl alcohol or a pharmaceutically acceptable salt, conjugate, hydrate, solvate, polymorph, or mixture thereof. In some embodiments, the HDCA or a pharmaceutically acceptable salt, conjugate, hydrate, solvate, polymorph, or mixture thereof, is present in the pharmaceutical formulation in an amount effective to result in a serum concentration of HDCA or the pharmaceutically acceptable salt, conjugate, hydrate, solvate, polymorph, or mixture thereof in the mammal in a range of from 1 mM to 1 M.

In some embodiments, a bile acid of the pharmaceutical formulation comprises DCA or a pharmaceutically acceptable salt, conjugate, hydrate, solvate, polymorph, or mixture thereof, and a terpene of the pharmaceutical formulation comprises S-perillyl alcohol or a pharmaceutically acceptable salt, conjugate, hydrate, solvate, polymorph, or mixture thereof. In some embodiments, the pharmaceutical formulation comprises DCA or the pharmaceutically acceptable salt, conjugate, hydrate, solvate, polymorph, or mixture thereof, in an amount effective to result in a serum concentration of DCA or the pharmaceutically acceptable salt, conjugate, hydrate, solvate, polymorph, or mixture thereof in the mammal in a range of from to 1 mM to 1 M.

In some embodiments, a terpene of the pharmaceutical formulation comprises D-limonene or a pharmaceutically acceptable salt, conjugate, hydrate, solvate, polymorph, or mixture thereof, in an amount effective to result in a serum concentration of D-limonene or the pharmaceutically acceptable salt, conjugate, hydrate, solvate, polymorph, or mixture thereof in the mammal in a range of from 1 mM to 1 M.

In some embodiments, a terpene of the pharmaceutical formulation comprises S-perillic acid or a pharmaceutically acceptable salt, conjugate, hydrate, solvate, polymorph, or mixture thereof, in an amount effective to result in a serum concentration of S-perillic acid or the pharmaceutically acceptable salt, conjugate, hydrate, solvate, polymorph, or mixture thereof in the mammal in a range of from 1 mM to 1 M.

In some embodiments, a terpene of the pharmaceutical formulation comprises 5-perillyl alcohol or a pharmaceutically acceptable salt, conjugate, hydrate, solvate, polymorph, or mixture thereof, in an amount effective to result in a serum concentration of S-perillic acid or the pharmaceutically acceptable salt, conjugate, hydrate, solvate, polymorph, or mixture thereof in the mammal in a range of from 1 mM to 1 M.

Some embodiments of the present invention provide a method of treating atherosclerosis in a mammal comprising administering to a mammal a pharmaceutical formulation comprising a bile acid or a pharmaceutically acceptable salt, conjugate, hydrate, solvate, polymorph, or mixture thereof; and a terpene or a pharmaceutically acceptable salt, conjugate, hydrate, solvate, polymorph, or mixture thereof. The formulation is administered in an amount effective to result in an amount of emulsification of an atherosclerotic plaque in an artery of the mammal sufficient to result in regression of the plaque.

In some Embodiments, a bile acid of the administered pharmaceutical formulation comprises HDCA, or a pharmaceutically acceptable salt, conjugate, hydrate, solvate, or polymorph thereof; deoxycholic acid (DCA), or a pharmaceutically acceptable salt, conjugate, hydrate, solvate, or polymorph thereof; or a mixture thereof.

In some Embodiments, a terpene of the administered pharmaceutical formulation comprises D-limonene, or a pharmaceutically acceptable salt, conjugate, hydrate, solvate, or polymorph thereof; S-perillic acid, or a pharmaceutically acceptable salt, conjugate, hydrate, solvate, or polymorph thereof; S-perillyl alcohol, or a pharmaceutically acceptable salt, conjugate, hydrate, solvate, or polymorph thereof; or a mixture thereof.

In some Embodiments, a bile acid of the administered pharmaceutical formulation comprises HDCA or a pharmaceutically acceptable salt, conjugate, hydrate, solvate, polymorph, or mixture thereof, and a terpene of the administered pharmaceutical formulation comprises D-limonene or a pharmaceutically acceptable salt, conjugate, hydrate, solvate, polymorph, or mixture thereof.

In some Embodiments, a bile acid of the administered pharmaceutical formulation comprises DCA or a pharmaceutically acceptable salt, conjugate, hydrate, solvate, polymorph, or mixture thereof, and a terpene of the administered pharmaceutical formulation comprises D-limonene or a pharmaceutically acceptable salt, conjugate, hydrate, solvate, polymorph, or mixture thereof.

In some Embodiments, a bile acid of the administered pharmaceutical formulation comprises HDCA or a pharmaceutically acceptable salt, conjugate, hydrate, solvate, polymorph, or mixture thereof, and a terpene of the administered pharmaceutical formulation comprises S-perillic acid or a pharmaceutically acceptable salt, conjugate, hydrate, solvate, polymorph, or mixture thereof.

In some Embodiments, a bile acid of the administered pharmaceutical formulation comprises DCA or a pharmaceutically acceptable salt, conjugate, hydrate, solvate, polymorph, or mixture thereof, and a terpene of the administered pharmaceutical formulation comprises S-perillic acid or a pharmaceutically acceptable salt, conjugate, hydrate, solvate, polymorph, or mixture thereof.

In some Embodiments, a bile acid of the administered pharmaceutical formulation comprises HDCA or a pharmaceutically acceptable salt, conjugate, hydrate, solvate, polymorph, or mixture thereof, and a terpene of the administered pharmaceutical formulation comprises S-perillyl alcohol or a pharmaceutically acceptable salt, conjugate, hydrate, solvate, polymorph, or mixture thereof. In some embodiments, the pharmaceutical formulation comprises HDCA or the pharmaceutically acceptable salt, conjugate, hydrate, solvate, polymorph, or mixture thereof, in an amount effective to result in a serum concentration of HDCA or the pharmaceutically acceptable salt, conjugate, hydrate, solvate, polymorph, or mixture thereof in the mammal in a range of from to 1 mM to 1 M.

In some Embodiments, a bile acid of the administered pharmaceutical formulation comprises DCA or a pharmaceutically acceptable salt, conjugate, hydrate, solvate, polymorph, or mixture thereof, and a terpene of the administered pharmaceutical formulation comprises S-perillyl alcohol or a pharmaceutically acceptable salt, conjugate, hydrate, solvate, polymorph, or mixture thereof. In some embodiments, the pharmaceutical formulation comprises DCA or the pharmaceutically acceptable salt, conjugate, hydrate, solvate, polymorph, or mixture thereof, in an amount effective to result in a serum concentration of DCA or the pharmaceutically acceptable salt, conjugate, hydrate, solvate, polymorph, or mixture thereof in the mammal in a range of from to 1 mM to 1 M.

In some embodiments, the administered pharmaceutical formulation comprises D-limonene or a pharmaceutically acceptable salt, conjugate, hydrate, solvate, polymorph, or mixture thereof, in an amount effective to result in a serum concentration of D-limonene or the pharmaceutically acceptable salt, conjugate, hydrate, solvate, polymorph, or mixture thereof in the mammal in a range of from 1 mM to 1 M.

In some embodiments, the administered pharmaceutical formulation comprises S-perillic acid or a pharmaceutically acceptable salt, conjugate, hydrate, solvate, polymorph, or mixture thereof, in an amount effective to result in a serum concentration of S-perillic acid or the pharmaceutically acceptable salt, conjugate, hydrate, solvate, polymorph, or mixture thereof in the mammal in a range of from 1 mM to 1 M.

In some embodiments, the administered pharmaceutical formulation comprises 5-perillyl alcohol or a pharmaceutically acceptable salt, conjugate, hydrate, solvate, polymorph, or mixture thereof, in an amount effective to result in a serum concentration of S-perillic acid or the pharmaceutically acceptable salt, conjugate, hydrate, solvate, polymorph, or mixture thereof in the mammal in a range of from 1 mM to 1 M.

In some embodiments, a terpene of the administered pharmaceutical formulation comprises D-limonene or a pharmaceutically acceptable salt, conjugate, hydrate, solvate, polymorph, or mixture thereof, and the pharmaceutical formulation is administered in a dose comprising an amount of D-limonene in a range of from 1 mg/kg/day to 20 g/kg/day.

In some embodiments, a terpene of the administered pharmaceutical formulation comprises S-perillic acid or a pharmaceutically acceptable salt, conjugate, hydrate, solvate, polymorph, or mixture thereof, and the pharmaceutical formulation is administered in a dose comprising an amount of S-perillic acid in a range of from 1 mg/kg/day to 20 g/kg/day.

In some embodiments, a terpene of the administered pharmaceutical comprises S-perillyl alcohol or a pharmaceutically acceptable salt, conjugate, hydrate, solvate, polymorph, or mixture thereof, and the pharmaceutical formulation is administered in a dose comprising an amount of S-perillyl alcohol in a range of from 1 mg/kg/day to 20 g/kg/day.

Some embodiments of the present invention provide a pharmaceutical formulation, for treating atherosclerosis in a mammal, comprising an active ingredient consisting essentially of at least one member selected from the group consisting essentially of a bile acid or a pharmaceutically acceptable salt, conjugate, hydrate, solvate, polymorph, or mixture thereof; and a terpene or a pharmaceutically acceptable salt, conjugate, hydrate, solvate, polymorph, or mixture thereof, and the formulation comprises an amount of active ingredient effective to result in an amount of emulsification of an atherosclerotic plaque in an artery of the mammal sufficient to result in regression of the plaque.

In some embodiments, the active ingredient consists essentially of DCA or a pharmaceutically acceptable salt, conjugate, hydrate, solvate, polymorph, or mixture thereof, and D-limonene or a pharmaceutically acceptable salt, conjugate, hydrate, solvate, polymorph, or mixture thereof.

In some embodiments, the active ingredient consists essentially of DCA or a pharmaceutically acceptable salt, conjugate, hydrate, solvate, polymorph, or mixture thereof, and S-perillic acid or a pharmaceutically acceptable salt, conjugate, hydrate, solvate, polymorph, or mixture thereof.

In some embodiments, the active ingredient consists essentially of DCA or a pharmaceutically acceptable salt, conjugate, hydrate, solvate, polymorph, or mixture thereof, and S-perillyl alcohol or a pharmaceutically acceptable salt, conjugate, hydrate, solvate, polymorph, or mixture thereof.

In some embodiments, the active ingredient consists essentially of HDCA or a pharmaceutically acceptable salt, conjugate, hydrate, solvate, polymorph, or mixture thereof, and D-limonene or a pharmaceutically acceptable salt, conjugate, hydrate, solvate, polymorph, or mixture thereof.

In some embodiments, the active ingredient consists essentially of HDCA or a pharmaceutically acceptable salt, conjugate, hydrate, solvate, polymorph, or mixture thereof, and S-perillic acid or a pharmaceutically acceptable salt, conjugate, hydrate, solvate, polymorph, or mixture thereof.

In some embodiments, the active ingredient consists essentially of HDCA or a pharmaceutically acceptable salt, conjugate, hydrate, solvate, polymorph, or mixture thereof, and S-perillyl alcohol or a pharmaceutically acceptable salt, conjugate, hydrate, solvate, polymorph, or mixture thereof.

In some embodiments, the active ingredient consists essentially of D-limonene or a pharmaceutically acceptable salt, conjugate, hydrate, solvate, polymorph, or mixture thereof, and the pharmaceutical formulation comprises the active ingredient in an amount effective to result in a serum concentration of D-limonene or the pharmaceutically acceptable salt, conjugate, hydrate, solvate, polymorph, or mixture thereof in the mammal in a range of from 1 mM to 1 M mM.

In some embodiments, the active ingredient consists essentially of S-perillic acid or a pharmaceutically acceptable salt, conjugate, hydrate, solvate, polymorph, or mixture thereof, and the pharmaceutical formulation comprises the active ingredient in an amount effective to result in a serum concentration of active ingredient in the mammal in a range of from 1 mM to 1 M.

In some embodiments, the active ingredient consists essentially of S-perillyl alcohol or a pharmaceutically acceptable salt, conjugate, hydrate, solvate, polymorph, or mixture thereof, and the pharmaceutical formulation comprises the active ingredient in an amount effective to result in a serum concentration of active ingredient in the mammal in a range of from 1 mM to 1 M.

Some embodiments of the present invention provide a method of treating atherosclerosis in a mammal comprising administering to a mammal a pharmaceutical formulation comprising an active ingredient consisting essentially of at least one member selected from the group consisting essentially of a bile acid or a pharmaceutically acceptable salt, conjugate, hydrate, solvate, polymorph, or mixture thereof, and a terpene or a pharmaceutically acceptable salt, conjugate, hydrate, solvate, polymorph, or mixture thereof. The pharmaceutical formulation is administered in an amount effective to result in an amount of emulsification of an atherosclerotic plaque in an artery of the mammal sufficient to result in regression of the plaque.

In some embodiments, the active ingredient of the administered pharmaceutical formulation consists essentially of DCA or a pharmaceutically acceptable salt, conjugate, hydrate, solvate, polymorph, or mixture thereof, and D-limonene or a pharmaceutically acceptable salt, conjugate, hydrate, solvate, polymorph, or mixture thereof.

In some embodiments, the active ingredient of the administered pharmaceutical formulation consists essentially of DCA or a pharmaceutically acceptable salt, conjugate, hydrate, solvate, polymorph, or mixture thereof, and S-perillic acid or a pharmaceutically acceptable salt, conjugate, hydrate, solvate, polymorph, or mixture thereof.

In some embodiments, the active ingredient of the administered pharmaceutical formulation consists essentially of DCA or a pharmaceutically acceptable salt, conjugate, hydrate, solvate, polymorph, or mixture thereof, and S-perillyl alcohol or a pharmaceutically acceptable salt, conjugate, hydrate, solvate, polymorph, or mixture thereof.

In some embodiments, the active ingredient of the administered pharmaceutical formulation consists essentially of HDCA or a pharmaceutically acceptable salt, conjugate, hydrate, solvate, polymorph, or mixture thereof, and D-limonene or a pharmaceutically acceptable salt, conjugate, hydrate, solvate, polymorph, or mixture thereof.

In some embodiments, the active ingredient of the administered pharmaceutical formulation consists essentially of HDCA or a pharmaceutically acceptable salt, conjugate, hydrate, solvate, polymorph, or mixture thereof, and S-perillic acid or a pharmaceutically acceptable salt, conjugate, hydrate, solvate, polymorph, or mixture thereof.

In some embodiments, the active ingredient of the administered pharmaceutical formulation consists essentially of HDCA or a pharmaceutically acceptable salt, conjugate, hydrate, solvate, polymorph, or mixture thereof, and S-perillyl alcohol or a pharmaceutically acceptable salt, conjugate, hydrate, solvate, polymorph, or mixture thereof.

In some embodiments, the active ingredient of the administered pharmaceutical formulation consists essentially of D-limonene or a pharmaceutically acceptable salt, conjugate, hydrate, solvate, polymorph, or mixture thereof, and the pharmaceutical formulation comprises an amount of active ingredient effective to result in a serum concentration of active ingredient in the mammal in a range of from 1 mM to 1 M.

In some embodiments, the active ingredient of the administered pharmaceutical formulation consists essentially of S-perillic acid or a pharmaceutically acceptable salt, conjugate, hydrate, solvate, polymorph, or mixture thereof, and the pharmaceutical formulation comprises an amount of active ingredient effective to result in a serum concentration of active ingredient in the mammal in a range of from 1 mM to 1 M.

In some embodiments, the active ingredient of the administered pharmaceutical formulation consists essentially of S-perillyl alcohol or a pharmaceutically acceptable salt, conjugate, hydrate, solvate, polymorph, or mixture thereof, and the pharmaceutical formulation comprises an amount of active ingredient effective to result in a serum concentration of active ingredient in the mammal in a range of from 1 mM to 1 M.

In some embodiments, the active ingredient of the administered pharmaceutical formulation consists essentially of D-limonene or a pharmaceutically acceptable salt, conjugate, hydrate, solvate, polymorph, or mixture thereof, and the pharmaceutical formulation is administered in a dose comprising an amount of active ingredient in a range of from 1 mg/kg/day to 20 g/kg/day.

In some embodiments, the active ingredient of the administered pharmaceutical formulation consists essentially S-perillic acid or a pharmaceutically acceptable salt, conjugate, hydrate, solvate, polymorph, or mixture thereof, and the pharmaceutical formulation is administered in a dose comprising an amount active ingredient in a range of from 1 mg/kg/day to 20 g/kg/day.

In some embodiments, the active ingredient of the administered pharmaceutical formulation consists essentially of S-perillyl alcohol or a pharmaceutically acceptable salt, conjugate, hydrate, solvate, polymorph, or mixture thereof, and the pharmaceutical formulation is administered in a dose comprising an amount of active ingredient in a range of from 1 mg/kg/day to 20 g/kg/day.

In some embodiments, the pharmaceutical formulation comprises a permeability enhancer comprising at least one of a non-ionic detergent, an ionic detergent, and a zwitterionic detergent.

In some embodiments, the administering comprises performing at least one of iontophoresis, electroporation, sonophoresis, thermal poration, microneedle treatment, and dermabrasion.

In some embodiments, the pharmaceutical formulation is administered intravenously.

In some embodiments, the pharmaceutical formulation is administered intra-arterially.

In some embodiments, the pharmaceutical formulation is administered orally.

In some embodiments, the pharmaceutical formulation is administered sublingually.

In some embodiments, the pharmaceutical formulation is administered transdermally.

In some embodiments, the pharmaceutical formulation is administered via an implantable device.

In some embodiments, the pharmaceutical formulation is administered by injection.

In some embodiments, the pharmaceutical formulation is administered transmucosally.

In some embodiments, the pharmaceutical formulation further comprises a statin.

In some embodiments, the pharmaceutical formulation further comprises a liposome, wherein the liposome carries at least one of the bile acid or the pharmaceutically acceptable salt, conjugate, hydrate, solvate, polymorph, or mixture thereof and the terpene or the pharmaceutically acceptable salt, conjugate, hydrate, solvate, polymorph, or mixture thereof.

In some embodiments, the level of DCA in the systemic circulation of the mammal is sustained for a period of at least two hours. As used herein, “systemic circulation” refers to the entirety of components carried along with oxygenated blood by the cardiovascular system as it carries oxygenated blood away from the heart, to the body, and returns deoxygenated blood back to the heart, such as serum, blood plasma, blood cells, red blood cells, white blood cells, antibodies, proteins, nucleic acids, and immune cells.

Certain embodiments of the present invention provide a pharmaceutical formulation, for treating atherosclerosis in a mammal, comprising a bile acid or a pharmaceutically acceptable salt, conjugate, hydrate, solvate, polymorph, or mixture thereof; and a terpene or a pharmaceutically acceptable salt, conjugate, hydrate, solvate, polymorph, or mixture thereof.

In certain embodiments, the bile acid comprises HDCA or a pharmaceutically acceptable salt, conjugate, hydrate, solvate, polymorph, or mixture thereof.

In certain embodiments, the bile acid comprises DCA or a pharmaceutically acceptable salt, conjugate, hydrate, solvate, polymorph, or mixture thereof.

In certain embodiments, the terpene comprises D-limonene or a pharmaceutically acceptable salt, conjugate, hydrate, solvate, polymorph, or mixture thereof. In certain embodiments, the terpene comprises S-perillic acid or a pharmaceutically acceptable salt, conjugate, hydrate, solvate, polymorph, or mixture thereof.

In certain embodiments, the terpene comprises S-perillyl alcohol or a pharmaceutically acceptable salt, conjugate, hydrate, solvate, polymorph, or mixture thereof.

In certain embodiments, the bile acid comprises HDCA or a pharmaceutically acceptable salt, conjugate, hydrate, solvate, polymorph, or mixture thereof, and the terpene comprises D-limonene or a pharmaceutically acceptable salt, conjugate, hydrate, solvate, polymorph, or mixture thereof.

In certain embodiments, the bile acid comprises DCA or a pharmaceutically acceptable salt, conjugate, hydrate, solvate, polymorph, or mixture thereof, and the terpene comprises D-limonene or a pharmaceutically acceptable salt, conjugate, hydrate, solvate, polymorph, or mixture thereof.

In certain embodiments, the bile acid comprises HDCA or a pharmaceutically acceptable salt, conjugate, hydrate, solvate, polymorph, or mixture thereof, and the terpene comprises S-perillic acid or a pharmaceutically acceptable salt, conjugate, hydrate, solvate, polymorph, or mixture thereof.

In certain embodiments, the bile acid comprises DCA or a pharmaceutically acceptable salt, conjugate, hydrate, solvate, polymorph, or mixture thereof, and the terpene comprises S-perillic acid or a pharmaceutically acceptable salt, conjugate, hydrate, solvate, polymorph, or mixture thereof.

In certain embodiments, the bile acid comprises HDCA or a pharmaceutically acceptable salt, conjugate, hydrate, solvate, polymorph, or mixture thereof, and the terpene comprises S-perillyl alcohol or a pharmaceutically acceptable salt, conjugate, hydrate, solvate, polymorph, or mixture thereof.

In certain embodiments, the bile acid comprises DCA or a pharmaceutically acceptable salt, conjugate, hydrate, solvate, polymorph, or mixture thereof, and the terpene comprises S-perillyl alcohol or a pharmaceutically acceptable salt, conjugate, hydrate, solvate, polymorph, or mixture thereof.

In certain embodiments, the terpene comprises D-limonene or a pharmaceutically acceptable salt, conjugate, hydrate, solvate, polymorph, or mixture thereof, in an amount effective to result in a serum concentration of D-limonene or the pharmaceutically acceptable salt, conjugate, hydrate, solvate, polymorph, or mixture thereof in the mammal in a range of from 1 mM to 1 M.

In certain embodiments, the terpene comprises S-perillic acid or a pharmaceutically acceptable salt, conjugate, hydrate, solvate, polymorph, or mixture thereof, in an amount effective to result in a serum concentration of S-perillic acid or the pharmaceutically acceptable salt, conjugate, hydrate, solvate, polymorph, or mixture thereof in the mammal in a range of from to 1 mM to 1 M.

In certain embodiments, the terpene comprises S-perillic acid or a pharmaceutically acceptable salt, conjugate, hydrate, solvate, polymorph, or mixture thereof, in an amount effective to result in a systemic concentration of S-perillic acid or the pharmaceutically acceptable salt, conjugate, hydrate, solvate, polymorph, or mixture thereof in the mammal in a range of from 1 mM to 1 M.

Some embodiments of the present invention provide a drug eluting stent comprising an intravascular stent; and pharmaceutical formulation, for treating atherosclerosis in a mammal, comprising a bile acid or a pharmaceutically acceptable salt, conjugate, hydrate, solvate, polymorph, or mixture thereof; and a terpene or a pharmaceutically acceptable salt, conjugate, hydrate, solvate, polymorph, or mixture thereof in or on the stent, wherein the formulation is in an amount effective to result in regression of an atherosclerotic plaque in an artery of the mammal.

Certain embodiments of the present invention provide a drug eluting stent comprising an intravascular stent; and a pharmaceutical formulation, for treating atherosclerosis in a mammal, comprising a bile acid or a pharmaceutically acceptable salt, conjugate, hydrate, solvate, polymorph, or mixture thereof; and a terpene or a pharmaceutically acceptable salt, conjugate, hydrate, solvate, polymorph, or mixture thereof in or on the stent.

In some embodiments, pharmaceutical formulations comprising a combination of at least two bile acid, terpene, saponin, and/or detergent atherosclerotic plaque emulsifiers are administered to a mammal at doses effective to result in plaque regression, and the dose of each individual emulsifier in the combination can be lower than a dose that is effective to result in plaque regression when the emulsifier is administered alone. In some embodiments, the pharmaceutical formulation comprises a lipase. In some embodiments, the lipase comprises an cholesteryl ester hydrolase. In some embodiments, the lipase comprises an cholesterol esterase. In some embodiments, the pharmaceutical formulation further comprises at least one of a lysyl oxidase and a lysyl oxidase agonist.

Some embodiments provide a drug eluting stent comprising: an intravascular stent; and a pharmaceutical formulation in or on the stent, wherein the pharmaceutical formulation comprises a bile acid or a pharmaceutically acceptable salt, conjugate, hydrate, solvate, polymorph, or mixture thereof and a terpene or a pharmaceutically acceptable salt, conjugate, hydrate, solvate, polymorph, or mixture thereof wherein the formulation is in an amount effective to result in regression of an atherosclerotic plaque in an artery of the mammal.

Some embodiments provide a drug eluting stent comprising: an intravascular stent; and a pharmaceutical formulation in or on the stent, wherein the pharmaceutical formulation comprises a bile acid or a pharmaceutically acceptable salt, conjugate, hydrate, solvate, polymorph, or mixture thereof and a terpene or a pharmaceutically acceptable salt, conjugate, hydrate, solvate, polymorph, or mixture thereof

Some embodiments of the present invention provide a method of treating atherosclerosis in a mammal comprising administering to a mammal a pharmaceutical formulation comprising: a terpene or a pharmaceutically acceptable salt, conjugate, hydrate, solvate, polymorph, or mixture thereof wherein the formulation is in an amount effective to result in regression of an atherosclerotic plaque in an artery of the mammal.

In some embodiments, the terpene comprises D-limonene or a pharmaceutically acceptable salt, conjugate, hydrate, solvate, polymorph, or mixture thereof. In some embodiments, the terpene comprises S-perillic acid or a pharmaceutically acceptable salt, conjugate, hydrate, solvate, polymorph, or mixture thereof. In some embodiments, the terpene comprises S-perillyl alcohol or a pharmaceutically acceptable salt, conjugate, hydrate, solvate, polymorph, or mixture thereof. In some embodiments, the terpene comprises D-limonene or a pharmaceutically acceptable salt, conjugate, hydrate, solvate, polymorph, or mixture thereof, in an amount effective to result in a serum concentration of D-limonene or the pharmaceutically acceptable salt, conjugate, hydrate, solvate, polymorph, or mixture thereof in the mammal in a range of from 1 mM to 1 M. In some embodiments, the terpene comprises S-perillic acid or a pharmaceutically acceptable salt, conjugate, hydrate, solvate, polymorph, or mixture thereof, in an amount effective to result in a serum concentration of S-perillic acid or the pharmaceutically acceptable salt, conjugate, hydrate, solvate, polymorph, or mixture thereof in the mammal in a range of from 1 mM to 1 M. In some embodiments, the pharmaceutical formulation comprises S-perillyl alcohol or a pharmaceutically acceptable salt, conjugate, hydrate, solvate, polymorph, or mixture thereof, in an amount effective to result in a systemic concentration of S-perillic acid or the pharmaceutically acceptable salt, conjugate, hydrate, solvate, polymorph, or mixture thereof in the mammal in a range of from 1 mM to 1 M. In some embodiments, the terpene comprises D-limonene or a pharmaceutically acceptable salt, conjugate, hydrate, solvate, polymorph, or mixture thereof, and wherein the pharmaceutical formulation is administered in a dose comprising an amount of D-limonene in a range of from 1 mg/kg/day to 20 g/kg/day. In some embodiments, the terpene comprises S-perillic acid or a pharmaceutically acceptable salt, conjugate, hydrate, solvate, polymorph, or mixture thereof, and wherein the pharmaceutical formulation is administered in a dose comprising an amount of S-perillic acid in a range of from 1 mg/kg/day to 20 g/kg/day. In some embodiments, the terpene comprises S-perillyl alcohol or a pharmaceutically acceptable salt, conjugate, hydrate, solvate, polymorph, or mixture thereof, and wherein the pharmaceutical formulation is administered in a dose comprising an amount of S-perillyl alcohol in a range of from 1 mg/kg/day to 20 g/kg/day.

As used herein, “combinations” of emulsifiers provided in certain embodiments means the emulsifiers are administered simultaneously, sequentially, or both.

Accordingly, in some embodiments there is provided, a method, of treating atherosclerosis in a patient, comprising: administering, across an epithelium of a patient, a pharmaceutical formulation comprising an emulsifier; enhancing a permeability of the epithelium to the emulsifier with a permeability enhancer; wherein enhancing the permeability of the epithelium is effective to result in passage of the emulsifier across the epithelium and into the patient's systemic circulation; wherein the passage of the emulsifier across the epithelium results in sustained levels of the emulsifier in the patient's systemic circulation that are therapeutically effective to result in regression of an atherosclerotic plaque.

In some embodiments, the sustained levels of the emulsifier in the systemic circulation are greater than 50 μM. In some embodiments, the sustained levels of the emulsifier in the systemic circulation are in a range between about 50 μM and about 600 μM. In some embodiments, the sustained levels of the emulsifier in the systemic circulation are in a range between about 100 μM and about 300 μM.

In some embodiments, the emulsifier comprises at least one of a bile acid, a saponin, a detergent, or pharmaceutically acceptable salts, conjugates, hydrates, solvates, polymorphs, or mixtures thereof. In some embodiments, the emulsifier comprises a bile acid, or pharmaceutically acceptable salts, conjugates, hydrates, solvates, polymorphs, or mixtures thereof.

In some embodiments, the emulsifier comprises deoxycholic acid.

In some embodiments, the sustained levels of the deoxycholic acid in the systemic circulation are greater than 50 μM. In some embodiments, the sustained levels of the deoxycholic acid in the systemic circulation are in a range between about 50 μM and about 600 μM. In some embodiments, the sustained levels of the deoxycholic acid in the systemic circulation are in a range between about 100 μM and about 300 μM.

In some embodiments, the emulsifier comprises a mixture of ursodeoxycholic acid and deoxycholic acid in substantially equimolar amounts. In some embodiments, the emulsifier comprises hyodeoxycholic acid. In some embodiments, the sustained levels of the hyodeoxycholic acid in the systemic circulation are greater than about 50 μM. In some embodiments, the sustained levels of the hyodeoxycholic acid in the systemic circulation are in a range from about 50 μM to about 600 μM. In some embodiments, the sustained levels of the hyodeoxycholic acid in the systemic circulation are in a range from about 100 μM to about 300 μM.

In some embodiments, the permeability enhancer comprises at least one of a non-ionic detergent, an ionic detergent, and a zwitterionic detergent. In some embodiments, the permeability enhancer comprises at least one of iontophoresis, electroporation, sonophoresis, thermal poration, microneedle treatment, and dermabrasion.

In some embodiments, the pharmaceutical formation is administered intravenously. In some embodiments, the pharmaceutical formation is administered intra-arterially. In some embodiments, the pharmaceutical formation is administered orally. In some embodiments, the pharmaceutical formation is administered sublingually. In some embodiments, the pharmaceutical formation is administered transdermally. In some embodiments, the pharmaceutical formation is administered via an implantable device. In some embodiments, the pharmaceutical formation is administered by injection. In some embodiments, the pharmaceutical formation is administered transmucosally.

In some embodiments, the method further comprises administering a statin either simultaneously or sequentially with the pharmaceutical formulation. In some embodiments, the pharmaceutical formulation further comprises the statin.

In some embodiments, there is provided a method of treating atherosclerosis in a patient comprising: administering a pharmaceutical formulation comprising an emulsifier in an amount effective achieve a concentration of the emulsifier in the systemic circulation of at least 50 μM; wherein the concentration of the emulsifier in the systemic circulation is sustained for a period of at least two hours; wherein the concentration of the emulsifier is effective to result in regression of an atherosclerotic plaque.

In some embodiments, the emulsifier comprises at least one of a bile acid, a saponin, a detergent, or pharmaceutically acceptable salts, conjugates, hydrates, solvates, polymorphs, or mixtures thereof. In some embodiments, the emulsifier comprises a bile acid, or pharmaceutically acceptable salts, conjugates, hydrates, solvates, polymorphs, or mixtures thereof.

In some embodiments, the sustained levels of the emulsifier in the systemic circulation are greater than 50 μM. In some embodiments, the sustained levels of the emulsifier in the systemic circulation are in a range between about 50 μM and about 600 μM. In some embodiments, the sustained levels of the emulsifier in the systemic circulation are in a range between about 100 μM and about 300 μM.

In some embodiments, the emulsifier comprises deoxycholic acid. In some embodiments, the sustained levels of the deoxycholic acid in the systemic circulation are greater than 50 μM. In some embodiments, the sustained levels of the deoxycholic acid in the systemic circulation are in a range between about 50 μM and about 600 μM. In some embodiments, the sustained levels of the deoxycholic acid in the systemic circulation are in a range between about 100 μM and about 300 μM.

In some embodiments, the emulsifier comprises a mixture of ursodeoxycholic acid and deoxycholic acid in substantially equimolar amounts.

In some embodiments, the emulsifier comprises hyodeoxycholic acid. In some embodiments, the sustained levels of the hyodeoxycholic acid in the systemic circulation are greater than about 50 μM. In some embodiments, the sustained levels of the hyodeoxycholic acid in the systemic circulation are in a range from about 50 μM to about 600 μM. In some embodiments, the sustained levels of the hyodeoxycholic acid in the systemic circulation are in a range from about 100 μM to about 300 μM.

In some embodiments, the method further comprises the use of a permeability enhancer. In some embodiments, the permeability enhancer comprises at least one of a non-ionic detergent, an ionic detergent, and a zwitterionic detergent. In some embodiments, the permeability enhancer comprises at least one of iontophoresis, electroporation, sonophoresis, thermal poration, microneedle treatment, and dermabrasion.

In some embodiments, the pharmaceutical formation is administered intravenously. In some embodiments, the pharmaceutical formation is administered intra-arterially. In some embodiments, the pharmaceutical formation is administered orally. In some embodiments, the pharmaceutical formation is administered sublingually. In some embodiments, the pharmaceutical formation is administered transdermally. In some embodiments, the pharmaceutical formation is administered via an implantable device. In some embodiments, the pharmaceutical formation is administered by injection. In some embodiments, the pharmaceutical formation is administered transmucosally.

In some embodiments, the method further comprises administering a statin either simultaneously or sequentially with the pharmaceutical formulation. In some embodiments, the pharmaceutical formulation further comprises the statin.

In some embodiments, there is provided a method of treating atherosclerosis in a patient comprising: administering a pharmaceutical formulation comprising an emulsifier in an amount effective achieve a concentration of the emulsifier in the systemic circulation of at least 50 μM at five minutes after onset of administration; wherein the concentration of the emulsifier in the systemic circulation is sustained above 50 μM for a period of at least two hours; and wherein the concentration of the emulsifier is effective to result in regression of an atherosclerotic plaque.

In some embodiments, the sustained levels of the emulsifier in the systemic circulation are greater than 50 μM. In some embodiments, the sustained levels of the emulsifier in the systemic circulation are in a range between about 50 μM and about 600 μM. In some embodiments, the sustained levels of the emulsifier in the systemic circulation are in a range between about 100 μM and about 300 μM.

### DETAILED DESCRIPTION OF THE INVENTIONS

One approach for treating atherosclerosis has been to use pharmaceuticals that inhibit the synthesis of cholesterol, an important component of LDL and of the lipid core of atherosclerotic plaques. Oxidized LDL provides, at least in part, the insult to the vessel wall that results in monocyte infiltration, their differentiation into macrophages, and the ensuing inflammatory reactions. Accordingly, statins are now a drug of choice in the treatment of atherosclerosis on the basis of their ability to decrease cholesterol synthesis by interfering with HMG-CoA reductase.

Other approaches for treating atherosclerosis involve methods for stabilizing plaques that reduce or eliminate the risk of plaque rupture and the attendant possibility of an acute coronary event. Still other approaches involve treating plaques locally with anti-thrombolytics to prevent complications arising from post-rupture clot formation, as disclosed, for example, in International Patent Application No. PCT/IN2006/000037 (Chandrasekar).

Despite the relatively widespread use of statins to treat atherosclerosis, these compounds only reduce but do not eliminate the risk of acute coronary events due to atherosclerotic plaque. There remains a need for methods of reducing plaque volume in patients, in essence to reverse the progression of atherosclerosis by effecting the regression of existing plaques.

U.S. Pat. No. 7,141,045 (Johansson et al.) discloses a method of dissolving a plaque by direct application of a dissolution fluid through an intravascular catheter. The dissolution fluid can include a variety of detergents, surfactants, and other solubilizing agents, in addition to enzymes, and metal ion chelators. While such an approach might be useful for acute treatment of known atherosclerotic lesions, it is seriously limited in its utility. First, the procedure is invasive, and can only be performed by a surgeon in an operating room situation. This necessarily means the procedure will be costly. Second, the treatment is only effective for known plaques reachable by catheter. Local treatment is therefore generally ineffective as a sole method for the systemic treatment of atherosclerotic plaques.

Accordingly, there remains a need for non-invasive, systemically effective compositions and treatments effective to result in solubilization and regression of atherosclerotic plaques, especially soft and/or vulnerable plaques. Results from prior studies, testing whether statins were effective to cause plaque regression, have been described as equivocal. For example, in the recently completed ASTEROID study (Nissen et al., (2006), JAMA 295: 1556-1565), experiments were designed to test whether 40 mg/day of rosuvastatin would be effective to result in a decrease in plaque volume, as evidenced by intravascular ultrasound imaging techniques. While the treatment was particularly effective at modulating LDL, HDL, and triglyceride levels, plaque volume after 2 years was only reduced by 8.5% (SD=13.7) in the most diseased segments of vessels examined, and by only 6.7% (SD=11.1) with respect to normalized total atheroma volume. Thus, statins are not particularly effective at producing significant reductions in plaque burden, even when provided at twice the normally prescribed dosage for a period of two years.

Some embodiments of the present invention provide pharmaceutical formulations comprising plaque emulsifiers, administered either systemically or locally, to dissolve plaque and result in plaque regression. Such emulsifiers include bile acids, bile salts, terpenes, saponins, detergents, and combinations thereof.

As used herein, an emulsion can comprise a mixture of two partially or completely immiscible liquids, one dispersed in the other. An emulsion can also comprise a colloid system in which both the dispersed phase and the dispersion medium comprise liquids and/or solids.

Bile acids are cholesterol-derived organic acids that have detergent properties. Bile acids play important roles physiologically in the digestion, absorption, transport, and secretion of lipids. Bile acids are involved in intestinal lipid digestion, by promoting fine emulsification of lipids, which enhances the exposure of lipids to lipid-digesting enzymes, such as pancreatic lipases. In addition to being direct emulsifiers of atherosclerotic plaque lipids, bile acids can also function to directly activate (e.g., allosteric effectors) lipases, such as cholesteryl ester hydrolase, that can be found in the arterial wall. In some embodiments, bile acids can emulsify short chain fatty acids released from atherosclerotic plaque lipid aggregates by lipase enzymatic activity.

Bile acids can be classified as primary or secondary bile acids, depending on whether they are synthesized de novo (primary) or are derived by subsequent chemical modification (secondary). Primary bile acids are produced by the liver and include cholic acid (3α, 7α, 12α,-trihydroxy-5β-cholanic acid) and chenodeoxycholic acid (3α, 7α,-dihydroxy-β-cholanic acid). Dehydroxylation of the primary bile acids, for example by intestinal bacteria, produces the more hydrophobic secondary bile acids, for example deoxycholic acid (3α, 12α,-dihydroxy-5β-cholanic acid), and lithocholic acid (3α-hydroxy-5β-cholanic acid). Together, the primary and secondary bile acids make up about 99% of the total bile acid pool in humans.

The role of circulating bile acid levels in the development of atherosclerosis is not clear in the prior art. Previous studies in animal model systems have suggested that lowering circulating levels of bile acids through the use of bile acid sequestrants lowers LDL levels and results in regression of atherosclerotic plaques (Wissler, J. Clin. Apher. 4: 52-58, 2006). The bile acid sequestrant, colesevelam HCl, has been shown to reduce LDL particle number and increase LDL particle size in patients with hypercholesterolemia (Rosenson, Atheroscl. 185: 327-330, 2006). Dietary supplements comprising bile acid polymeric organic bases have been shown to inhibit cholesterol rise and atherosclerotic plaque formation in chickens on a high cholesterol diet (Tennent et al., J. Lip. Res. 1: 469-473, 1960). Thus, collectively, the prior art suggests that decreasing circulating bile acid levels should be effective to reduce progression, or even promote regression of atherosclerotic plaques.

Contrary to these prior art studies, where reducing circulating levels of bile salts is predicted to slow or regress plaque, embodiments of the present disclosure teach formulations and methods that lead to a sustained increase in the level of bile acid and/or bile salt emulsifiers in the systemic circulation are effective to dissolve the lipid components of atherosclerotic plaque, and result in plaque regression. Experimental examples described below demonstrate that bile acid emulsifiers are effective to dissolve the lipid core of atherosclerotic plaques.

There are instances where the concentration of bile acids have been increased systemically. For example, it has been previously shown that feeding hyodeoxycholic acid (HDCA) to C57BL/6 LDL r-KO knockout mice (genetically predisposed to develop atherosclerosis) results in a reduced rate of formation of atherosclerotic plaque relative to mice not provided HDCA (Sehayek et al., J. Lip. Res. 42: 1250-1256, 2001). Plasma levels of wild-type mice, provided the same amount of dietary HDCA, ranged up to about 50 μM. However, there is no evidence that these levels were effective to result in plaque regression, as provided by certain embodiments described herein.

Primary biliary cirrhosis (PBC) is an inflammatory disease characterized by destruction of the small bile ducts within the liver, eventually leading to cirrhosis. While the cause of PBC is not precisely known, the presence of auto-antibodies in PBC patients suggests an autoimmune origin. Among the various symptoms that arise as a result of PBC, it is known that total plasma cholesterol tends to be elevated, by as much as 50%. Despite the increases in cholesterol levels, however, it appears that PBC patients are not at an increased risk of atherosclerosis. In addition, it has been shown that PBC patients have elevated levels of bile acids (Murphy et al., Gut 13: 201-206, 1972), with levels averaging about 200 μM, as compared to normal levels which are less than 10 μM. Some embodiments described herein are effective to mimic the high levels of bile salts observed in PBC patients, and in so doing are effective to result in atherosclerotic plaque regression.

In some embodiments, administration schedules of a pharmaceutical formulation comprising bile acid, terpene, saponin, and/or detergent atherosclerotic plaque emulsifiers effective to result in plaque regression involve administering the formulation once per day, twice per day, three times per day, four times per day, five times per day, six times per day, seven times per day, eight times per day, nine times per day, 10 times per day, 11 times per day, 12 times per day, 13 times per day, 14 times per day, 15 times per day, 16 times per day, 17 times per day, 18 times per day, 19 times per day, 20 times per day, 21 times per day, 22 times per day, 23 times per day, 24 times per day, and continuously. In some embodiments, daily or continuous administration of a pharmaceutical formulation of the present invention may comprise a period of at least one day, two days, three days, four days, five days, six days, seven days, two weeks, three weeks, one month, two months, three months, four months, five months, six months, seven months, eight months, nine months, 10 months, 11 months, one year, two years, three years, four years, and five years. In some embodiments, daily or continuous administration of the pharmaceutical formulation may be intermittent within an administration period, for instance, every other day, every third day, every fourth day, every fifth day, every sixth day, once a week, once every two weeks, once every three weeks, once a month, once every two months, once every three months, once every four months, once every five months, once every six months, once every seven months, once every eight months, once every nine months, once every 10 months, once every 11 months, and once a year.

In some embodiments, an effective dose of a pharmaceutical formulation results in elevated levels of bile acid, terpene, saponin, and/or detergent atherosclerotic plaque emulsifiers in the systemic circulation sustained for a period of, for instance, at least about one hour, about two hours, about three hours, about four hours, about five hours, about six hours, about seven hours, about eight hours, about nine hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, and about 24 hours.

In some embodiments, an effective dose of a pharmaceutical formulation comprising an emulsifier or a combination of emulsifiers results in a regression in a size, e.g. a diameter, a thickness, and/or a volume, in a range of from, for instance, about 1% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, and about 90% to about 100%.

In some embodiments, sustained levels of an effective dose of a pharmaceutical formulation comprising an emulsifier or a combination of emulsifiers of the present invention is effective to dissolve an amount of an insoluble and or aggregated plaque component of an atherosclerotic in a range of from, for instance, about 1% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, and about 90% to about 100%.

### Examples of Cholesterol Components of Atherosclerotic Plaques

In some embodiments, atherosclerotic plaques treated by the methods and pharmaceutical formulations of the present invention comprise at least one of cholesterol crystals, oleate cholesteryl esthers, linoleate cholesteryl esthers, and/or palmitate cholesteryl esthers in insoluble or aggregated form.

### Examples of Bile Acid Emulsifiers

As used herein, the term “bile acid” includes bile acids; pharmaceutically acceptable salts, conjugates, hydrates, solvates, derivatives, or polymorphs of bile acids; and mixtures thereof. Examples of bile acids useful in certain embodiments described herein can include, without limitation any naturally occurring or synthetically produced bile acid, salt, or conjugate thereof, having the ability to solubilize a lipid component of an atherosclerotic plaque. This can include cholic acid, chenodeoxycholic acid, deoxycholic acid, lithocholic acid, ursodeoxycholic acid, hyodeoxycholic acid, and any conjugate or pharmaceutically acceptable salt thereof.

In addition, bile acids useful in certain embodiments of formulations for use as described herein can include, without limitation: 1,3,12-trihydroxycholanoic acid; 1,3,7,12-tetrahydroxycholanoic acid; 3beta-hydroxy-delta 5-cholenic acid; 3 beta-hydroxychol-3-en-24-oic acid; 3′-isøthiocyanatobenzamidecholic acid; 3,12-dihydroxy-5-cholenoic acid; 3,4,7-trihydroxycholanoic acid; 3,6,12-trihydroxycholanoic acid; 3,7,12,23-tetrahydroxycholan-24-oic acid; 3,7,12-trihydroxy-7-methylcholanoic acid; 3,7,12-trihydroxycoprostanoic acid; 3,7,23-trihydroxycholan-24-oic acid; 3,7-dihydroxy-22,23-methylene-cholan-24-oic acid (2-sulfoethyl)amide; 3-((3-cholamidoρropyl)dimethylammonium)-1-propanesulfonate; 3-((3-deoxycholamidopropyl)dimethylammonio)-1-propane; 3-benzoylcholic acid; 3-hydroxy-5-cholen-24-oic acid 3-sulfate ester; 3-hydroxy-7-(hydroxyimino)cholanic acid; 3-ïodocholic acid; 7,12-dihydroxy-3-(2-(glucopyranosyl)acetyl)cholan-24-oic acid; 7,12-dihydroxy-3-oxocholanoic acid; allocholic acid; chapso; chol-3-en-24-oic acid; cholanic acid; sodium cholate; methyl cholate; benzyldimethylhexadecylammonium cholate; methyl 1,3-dihydroxycholan-24-oate; and trioctylmethylammonium cholate); cholic acid glucuronide; cholyl-coenzyme A; cholyl-lysylfluorescein; cholyldiglycylhistamine; cholylhistamine; cholylhydroxamic acid; cholylsarcosine; cholyltetraglycylhistamine; ciliatocholic acid; dehydrocholic acid (which includes FZ 560; Gallo-Merz; Gillazym; Hepavis; Mexase; progresin Retard; and spasmocanulase); 23-nordeoxycholic acid; 3,7-dioxocholanoic acid; 3-hydroxy-ρolydeoxycholic acid; 3-sulfodeoxycholic acid; 6-hydroxycholanoic acid; 6-methylmurideoxycholic acid; 7-ketodeoxycholic acid; 7-methyldeoxycholic acid; chenodeoxycholic acid; dehydrodeoxycholic acid; deoxycholyltyrosine; desoxybilianic acid; glycodeoxycholic acid; hyodeoxycholate-6-O-glucuronide; hyodeoxycholic acid; taurodeoxycholic Acid; and ursodeoxycholic acid; glycocholic acid; 3-hydroxy-5-cholenoylglycine; cholylglycylhistamine; cholylglycyltyrosine; glycodeoxycholic Acid; sulfolithocholylglycine; hemulcholic acid; 12-ketolithocholic acid; 24-norlithocholic acid; 3-dehydrolithocholylglycine; 3-hydroxy-6-cholen-24-oic acid; 3-hydroxy-7,12-diketocholanoic acid; 3-hydroxy-7-methylcholanoic acid; 3-ketolithocholic acid; 3-oxochol-4-en-24-oic acid; 3-oxocholan-24-oic acid; 4-azidophenacyl lithocholate; 7-ketolithocholic acid; BRL 39924A; glycolithocholic acid; lithocholate 3-O-glucuronide; lithocholyl-N-hydroxysuccinimide; methyl lithocholate; N-carbobenzoxy-N-lithocholyl-epsilon-lysine; N-epsilon-lithochoiyllysine; sulfolithocholic acid; and taurolithocholic acid; muricholic acid; N-(1,3,7,12-tetrahydroxycholan-24-oyl)-2-aminopropionic acid; N-(2-aminoethyl)-3,7,12-trihydroxycholan-24-amide; N-carboxymethyl)-N-(2-(bis(carboxymethyl)amino)ethyl)-3-(4-(N′-(2-((3,7,12-trihydroxycholan-24-oyl)araino)ethyl)(thioureido)ρhenyl)alanine; N-cholyl-2-fluoro-beta-alanine; norcholic acid; norursocholic acid; taurocholic acid; (N-(7-(nitrobenz-2-oxa-1,3-diazol-4-yl))-7-amino-3alpha,12alpha-dihydroxycholan-24-oyl)-2-aminoethanesulfonate; 23-seleno-25-homotaurocholic acid; 3,12-dihydroxy˜7˜oxocholanoyltaurine; 3-hydroxy-7-oxocholanoyltaurine; azidobenzamidotaurocholate; hexadecyltributylammonium taurocholate; tauro 1-hydroxycholic acid; tauro-3,7-dihydroxy-12-ketocholanoic acid; taurodehydrocholate; taurodeoxycholic acid; tauroglycocholic acid; taurolithocholic acid; tauromurichoUc acid; tauronorcholic acid); tetrahydroxy-5-cholan-24-oic acid; ursocholic acid; vulpecholic acid; bile acid sulfates; glycodeoxycholic acid; glycochenodeoxycholic acid; 7-oxoglycochenodeoxycholic acid; glycochenodeoxycholate-3-sulfate; glycohyodeoxycholic acid; tauro-7,12-dihydroxycholanoic acid; taurochenodeoxycholic acid; taurochenodeoxycholate-3-sulfate; taurochenodeoxycholate-7-sulfate; tauroursodeoxycholic acid; taurohyodeoxycholic acid; the includes: 23-methylursodeoxycholic acid; 24-norursodeoxycholic acid; 3,6-dihj^{?}droxy-6-methylcholanoic acid; 3,7-dihydroxy-20,22-methylenecholan-23-oic acid; 3,7-dihydroxy-22,23-methylenecholan-24-oic acid; 3,7-dihydroxy-7-ethylcholanoic acid; 3,7-dihydroxy-7-methylcholanoic acid; 3,7-dihydroxy-7-n-propylcholanoic acid; Bamet-UD2; diammhiebis(ursodeoxycholate(O,O′))ρIatinum(II); glycoursodeoxycholic acid; homoursodeoxycholic acid; HS 1030; HS 1183; isoursodeoxycholic acid; PABA-ursodeoxycholic acid; sarcosylsarcoursodeoxycholic acid; sarcoursodeoxycholic acid; ursodeoxycholate-3-sulfate; ursodeoxycholic acid 7-oleyl ester; ursodeoxycholic acid N-acetylglucosaminide; ursodeoxycholic acid-3-O-glucuronide; ursodeoxycholyl N-carboxymethylglycine; ursodeoxycholylcysteic acid; ursometh; 24-norchenodeoxycholic acid; 3,7-dihydroxy-12-oxocholanoic acid; 3,7-dihydroxy-24-norcholane-23-sulfonate; 3,7-dihydroxy-25-homocholane-25-sulfonate; 3,7-dihydroxychol-5-enoic acid; 3,7-dihydroxycholane-24-sulfonate; 3-glucosido-chenodeoxycholic acid; 3-oxo-7-hydroxychol-4-enoic acid; 6-ethylchenodeoxycholic acid; chenodeoxycholate sulfate conjugate; chenodeoxycholyltyrosine; glycochenodeoxycholic acid which includes: 7-oxoglycochenodeoxycholic acid and glycochenodeoxycholate-3-sulfate; homochenodeoxycholic acid; HS 1200; methyl 3,7-dihydroxychol-4-en-24-oate; methyl 3,7-dihydroxycholanate; N-(2-aminoethyl)-3,7-dihydroxycholan-24-amide; N-chenodeoxycholyl-2-fluoro-beta-alanine; sarcochenodeoxycholic acid; taurochenodeoxycholic acid; taurochenodeoxycholate-3-sulfate; taurochenodeoxycholate-7-sulfate; tauroursodeoxycholic acid.

In some embodiments, fatty acids conjugated to bile acids useful in certain embodiments of formulations for use as described herein can include, without limitation butyric acid, caproic acid, caprylic acid, lauric acid, myristic acid, palmitic acid, stearic acid, arachidic acid, behenic acid, oleic acid, linoleic acid, alpha-linolenic acid, arachidonic acid, eicosapentaenoic acid, docosahexaenoic acid, and euric acid.

### Examples of Serum and Systemic Circulation Concentrations of Bile Acid Emulsifiers

Serum and systemic circulation concentrations of a bile acid effective to result in regression of an atherosclerotic plaque may vary depending on a number of factors. Influential variables can include, for example, various chemical properties of one bile acid, as compared to another. For example different bile acids can differ in pKₐ solubility, molecular weight, etc., and these properties of a particular bile acid may affect how a patient metabolizes the bile acid, how much of the bile acid enters and remains in the systemic circulation of a mammal, and how effectively the bile acid emulsifies and dissolves atherosclerotic plaques.

Accordingly, in some embodiments of the present invention, a serum or a systemic circulation concentration of a bile acid effective to result in atherosclerotic plaque emulsification and regression may be in a range of from, for instance, about 1 μM to about 10 μM, about 5 μM to about 10 μM, about 10 μM to about 20 μM, about 20 μM to about 30 μM, about 30 μM to about 40 μM, about 40 about μM to about 50 μM, about 50 μM to about 60 μM, about 60 μM to about 70 μM, about 70 μM to about 80 μM, about 80 μM to about 90 μM, about 90 μM to about 100 μM, about 50 μM to about 600 μM, about 50 μM to about 100 μM, about 100 μM to about 300 μM, about 100 μM to about 550 μM, about 150 μM to about 500 μM, about 200 μM to about 450 μM, about 250 μM to about 400 μM, about 300 μM to about 350 μM, about 500 μM to about 600 μM, about 600 μM to about 700 μM, about 700 μM to about 800 μM, about 800 μM to about 900 μM, about 900 μM to about 1 mM, about 1 mM to about 100 mM, about 100 mM to about 200 mM, about 200 mM to about 300 mM, about 300 mM to about 400 mM, about 400 mM to about 500 mM, about 500 mM to about 600 mM, about 600 mM to about 700 mM, about 700 mM to about 800 mM, about 800 mM to about 900 mM, and about 900 mM to about 1 M.

### Examples of Bile Acid Doses

In some embodiments, a bile acid dose effective to result in atherosclerotic plaque emulsification and regression may be, in weight of administered bile acid per kilogram of mammal body weight per day (mg/kg/day), in a range of from, for instance, about 1 mg/kg/day to about 10 mg/kg/day, about 10 mg/kg/day to about 20 mg/kg/day, about 20 mg/kg/day to about 30 mg/kg/day, about 30 mg/kg/day to about 40 mg/kg/day, about 40 mg/kg/day to about 50 mg/kg/day, about 50 mg/kg/day to about 60 mg/kg/day, about 60 mg/kg/day to about 100 mg/kg/day, about 100 mg/kg/day to about 125 mg/kg/day, about 125 mg/kg/day to about 150 mg/kg/day, about 150 mg/kg/day to about 175 mg/kg/day, about 175 mg/kg/day to about 200 mg/kg/day, about 200 mg/kg/day to about 225 mg/kg/day, about 225 mg/kg/day to about 250 mg/kg/day, about 250 mg/kg/day to about 275 mg/kg/day, about 275 mg/kg/day to about 300 mg/kg/day, about 300 mg/kg/day to about 325 mg/kg/day, about 325 mg/kg/day to about 350 mg/kg/day, about 350 mg/kg/day to about 375 mg/kg/day, about 375 mg/kg/day to about 400 mg/kg/day, about 400 mg/kg/day to about 425 mg/kg/day, about 425 mg/kg/day to about 450 mg/kg/day, about 450 mg/kg/day to about 475 mg/kg/day, about 475 mg/kg/day to about 500 mg/kg/day, about 500 mg/kg/day to about 550 mg/kg/day, about 550 mg/kg/day to about 600 mg/kg/day, about 600 mg/kg/day to about 650 mg/kg/day, about 650 mg/kg/day to about 700 mg/kg/day, about 700 mg/kg/day to about 750 mg/kg/day, about 750 mg/kg/day to about 800 mg/kg/day, about 800 mg/kg/day to about 850 mg/kg/day, about 850 mg/kg/day to about 900 mg/kg/day, about 900 mg/kg/day to about 950 mg/kg/day, about 950 mg/kg/day to about 1 g/kg/day, about 1 g/kg/day to about 1.25 g/kg/day, about 1.25 g/kg/day to about 1.5 g/kg/day, about 1.5 g/kg/day to about 1.75 g/kg/day, about 1.75 g/kg/day to about 2 g/kg/day, about 2 g/kg/day to about 2.25 g/kg/day, about 2.25 g/kg/day to about 2.5 g/kg/day, about 2.5 g/kg/day to about 2.75 g/kg/day, about 2.750 g/kg/day to about 3 g/kg/day, about 3 g/kg/day to about 4 g/kg/day, about 4 g/kg/day to about 5 g/kg/day, about 5 g/kg/day to about 6 g/kg/day, about 6 g/kg/day to about 7 g/kg/day, about 7 g/kg/day to about 8 g/kg/day, about 8 g/kg/day to about 9 g/kg/day, about 9 g/kg/day to about 10 g/kg/day, about and 10 g/kg/day to about 20 g/kg/day.

### Examples of Terpene Emulsifiers

As used herein, the term “terpene” includes terpenes; pharmaceutically acceptable salts, conjugates, hydrates, solvates, derivatives, or polymorphs of terpenes; and mixtures thereof. Examples of terpene emulsifiers useful in certain embodiments described herein can include any naturally occurring or synthetically produced terpene, and/or terpene metabolite. Terpenes can be synthesized, and can also be found in nature, for instance, in plant essential oils. Terpenes comprise an isoprene building block, CH₂═C(CH₃)—CH═CH₂, and can comprise a basic molecular formula of (C₅H₈)ₙ and derivatives thereof, in which n is the number of linked isoprene units. The isoprene units of terpenes may be linked together “head to tail” to form linear chains or they may be arranged to form rings. As used herein, terpenes may comprise isoprene units modified with oxygen-containing compounds such as alcohols, aldehydes or ketones.

Hemiterpenes comprise a single isoprene unit, and an example of a hemiterpene is isoprene. Monoterpenes comprise two isoprene units, and examples of monoterpenes include menthol, gerinol, limonene, D-limonene, L-limonene, and terpinol. Metabolites of monopterpenes include S-perillic acid. Sesqueterpenes comprise three isoprene units, and examples of sesquiterpenes include farnesol. Diterpenes comprise four isoprene units, and are derived from geranylgeranyl phosphate. Examples of diterpenes include cafestol, kahweol, cembrene, and taxadiene, (precursor of Taxol). Diterpenes also form the basis for compounds such as retinol, retinal, and phytol. The herb sidiritis contains diterpenes. Sesterterpenes comprise five isoprene units. Triterpenes comprise six isoprene units, tetraterpenes contain eight isoprene units, and examples of tetraterpenes include provitamin A, acyclic lycopene, monocyclic carotene, and bicyclic alpha-carotene, and beta-carotene. Terpenes can also be used as permeability enhancers, effective to enhance the permeability of membranes or tissue to emulsifiers.

D-limonene and its derivatives, such as S-perillic acid and S-perillyl alcohol, comprise terpene emulsifiers of the present invention. It is known in the art that these compounds are quite safe and non-toxic for mammals. Experimental examples described below demonstrate that terpene emulsifiers are effective to dissolve the lipid core of atherosclerotic plaques.

### Examples of Serum and Systemic Circulation Concentrations of Terpene Emulsifiers

Serum and systemic circulation concentrations of a terpene emulsifier effective to result in atherosclerotic plaque regression may vary depending on a number of factors. Influential variables can include, for example, various chemical properties of one terpene, as compared to another. For example different terpenes can differ in pKₐ, solubility, molecular weight, etc., and these properties of a particular terpene may affect how a patient metabolizes the terpene, how much of the terpene enters and remains in the systemic circulation of a mammal, and how effectively the terpene emulsifies and dissolves atherosclerotic plaques.

Accordingly, in some embodiments of the present invention, a serum or a systemic circulation concentration of a terpene effective to result in atherosclerotic plaque emulsification and regression may be in a range of from, for instance, about 1 μM to about 10 μM, about 10 μM to about 20 μM, about 20 μM to about 30 μM, about 30 μM to about 40 μM, about 40 μM to about 50 μM, about 50 μM to about 60 μM, about 60 μM to about 100 μM, about 100 μM to about 125 μM, about 125 μM to about 150 μM, about 150 μM to about 175 μM, about 175 μM to about 200 μM, about 200 μM to about 225 μM, about 225 μM to about 250 μM, about 250 to 275 μM, about 275 μM to about 300 μM, about 300 μM to about 325 μM, about 325 μM to about 350 μM, about 350 μM to about 375 μM, about 375 μM to about 400 μM, about 400 μM to about 425 μM, about 425 μM to about 450 μM, about 450 μM to about 475 μM, about 475 μM to about 500 μM, about 500 μM to about 550 μM, about 550 μM to about 600 μM, about 600 μM to about 650 μM, about 650 μM to about 700 μM, about 700 μM to about 750 μM, about 750 μM to about 800 μM, about 800 μM to about 850 μM, about 850 μM to about 900 μM, about 900 μM to about 950 μM, 950 μM to about 1.0 mM, about 1 mM to about 10 mM, about 10 mM to about 20 mM, about 20 mM to about 30 mM, about 30 mM to about 40 mM, about 40 mM to about 50 mM, about 50 mM to about 60 mM, about 60 mM to about 100 mM, about 100 mM to about 125 mM, about 125 mM to about 150 mM, about 150 mM to about 175 mM, about 175 mM to about 200 mM, about 200 mM to about 225 mM, about 225 mM to about 250 mM, about 250 mM to about 275 mM, about 275 mM to about 300 mM, about 300 mM to about 325 mM, about 325 mM to about 350 mM, about 350 mM to about 375 mM, about 375 mM to about 400 mM, about 400 mM to about 425 mM, about 425 mM to about 450 mM, about 450 mM to about 475 mM, about 475 mM to about 500 mM, about 500 mM to about 550 mM, about 550 mM to about 600 mM, about 600 mM to about 650 mM, about 650 mM to about 700 mM, about 700 mM to about 750 mM, about 750 mM to about 800 mM, about 800 mM to about 850 mM, about 850 to about 900 mM, about 900 to about 950 mM, about 950 mM to about 1.0 M.

### Examples of Terpene Doses

In some embodiments, a terpene dose effective to result in atherosclerotic plaque emulsification and regression may be, in weight of administered terpene per kilogram of mammal body weight per day (mg/kg/day), in a range of from, for instance, about 1 mg/kg/day to about 10 mg/kg/day, about 10 mg/kg/day to about 20 mg/kg/day, about 20 mg/kg/day to about 30 mg/kg/day, about 30 mg/kg/day to about 40 mg/kg/day, about 40 mg/kg/day to about 50 mg/kg/day, about 50 mg/kg/day to about 60 mg/kg/day, about 60 mg/kg/day to about 100 mg/kg/day, about 100 mg/kg/day to about 125 mg/kg/day, about 125 mg/kg/day to about 150 mg/kg/day, about 150 mg/kg/day to about 175 mg/kg/day, about 175 mg/kg/day to about 200 mg/kg/day, about 200 mg/kg/day to about 225 mg/kg/day, about 225 mg/kg/day to about 250 mg/kg/day, about 250 mg/kg/day to about 275 mg/kg/day, about 275 mg/kg/day to about 300 mg/kg/day, about 300 mg/kg/day to about 325 mg/kg/day, about 325 mg/kg/day to about 350 mg/kg/day, about 350 mg/kg/day to about 375 mg/kg/day, about 375 mg/kg/day to about 400 mg/kg/day, about 400 mg/kg/day to about 425 mg/kg/day, about 425 mg/kg/day to about 450 mg/kg/day, about 450 mg/kg/day to about 475 mg/kg/day, about 475 mg/kg/day to about 500 mg/kg/day, about 500 mg/kg/day to about 550 mg/kg/day, about 550 mg/kg/day to about 600 mg/kg/day, about 600 mg/kg/day to about 650 mg/kg/day, about 650 mg/kg/day to about 700 mg/kg/day, about 700 mg/kg/day to about 750 mg/kg/day, about 750 mg/kg/day to about 800 mg/kg/day, about 800 mg/kg/day to about 850 mg/kg/day, about 850 mg/kg/day to about 900 mg/kg/day, about 900 mg/kg/day to about 950 mg/kg/day, about 950 mg/kg/day to about 1 g/kg/day, about 1 g/kg/day to about 1.25 g/kg/day, about 1.25 g/kg/day to about 1.5 g/kg/day, about 1.5 g/kg/day to about 1.75 g/kg/day, about 1.75 g/kg/day to about 2 g/kg/day, about 2 g/kg/day to about 2.25 g/kg/day, about 2.25 g/kg/day to about 2.5 g/kg/day, about 2.5 g/kg/day to about 2.75 g/kg/day, about 2.750 g/kg/day to about 3 g/kg/day, about 3 g/kg/day to about 4 g/kg/day, about 4 g/kg/day to about 5 g/kg/day, about 5 g/kg/day to about 6 g/kg/day, about 6 g/kg/day to about 7 g/kg/day, about 7 g/kg/day to about 8 g/kg/day, about 8 g/kg/day to about 9 g/kg/day, about 9 g/kg/day to about 10 g/kg/day, about and 10 g/kg/day to about 20 g/kg/day.

### Examples of Saponin Emulsifiers

As used herein, the term “saponin” includes saponins; pharmaceutically acceptable salts, conjugates, hydrates, solvates, derivatives, or polymorphs of saponins; and mixtures thereof. Saponins are naturally occurring compounds predominantly derived from plants, and can have detergent properties. The name saponin is derived from the soapwort plant (Saponaria) traditionally used in making a type of soap. Saponins are the glycosides of 27 carbon steroids or 30 carbon triterpenes. Removal of the sugar moiety from a saponin by hydrolysis yields the aglycone, sapogenin. Triterpenoid saponins are generally acid, and steroid saponins are generally neutral.

Steroid saponins include three classes of compounds, the cholestanol, furostanol, and spirostanol saponins Examples of furostanol saponins can include, proto-isoeruboside-B and isoeruboside-B, as well as saponins derived, for example, from Ruscus aculeatus, Tacca chantrieri, Solanum hispidum, Dioscorea polygonoides, Tribulus terrestris, and Lilium candidum. Other steroid saponins can include those derived from Saponaria officinalis, Yucca schidigera, and Chlorogalum pomeridianum.

Examples of triterpenoid saponins can include those of the fusidane-lanostante group, cyclopassiflosides, cycloglobiseposides, cycloartanes, dammaranes (e.g., bacopasaponin and jujubogenin), lupanes (e.g., quadranosides), oleananes (e.g., maesapinin), ligatosides, sandrosaponins, pedunsaponins), vulgarsaponin, peduncularisaponin, petersaponin, araliasaponin, assamsaponin, eupteleasaponin, herniariasaponin, jeosaponin, meliltussaponin, ursanes (e.g., randisaponins), brevicuspisaponin, ursolic acid, and indicasaponin. Triterpenoids can also be derived from Quillaja saponaria, as well as those derived from grapes.

Saponins have been identified in plants and animals including, for example, and without being limiting, agave, Agrostemma Githago, alfalfa, aloe, Alfombrilla, Anadenanthera peregrine, amaranth, Angelica sinesis, Aralia chinesis, Aralia manshurica, asparagus, Astragalus membranaceus, buckeyes soapwart, Bacopa monnieri, broomweed, Boussingaultia sp., Bupleurum chinense, Calendula officinalis, Capsicum sp., Christmas Rose, chickweed, chickpeas, Chlorophytum sp., Chlorogalum sp., corn cockle, Codonopsis pilosula, horse chestnuts, curcurbit, Daisies, Dioscorea sp, Drymaria arenaroides, Digitalis sp., Echinodermata, Elecampane, Elutherococcus senticosus, fenugreek, goldenrod, gotu kola, grape skin, Glycyrrhiza glabra, Gymnema sylvestre, Gymnostemma Pentaphyllum, Gypsophila sp., hawthorn, Helleborus niger, Honeylocust, jiaogulan, licorice, lungwort, mullein, Medicago sativa, Cicer arietinum olives, onion, pannax (Koren Ginseng), Platycodon sp, Platycodon grandiflorum, Polygala tenuifola, Quillaja saponaria, quinoa, Phytolacca americana, rambutan, Salvia sp., soapberry, Saponaria sp., Schizandra chinensis, shallots, southern pea, soybean, Tribulus terrestris, Tuberous cucurbit species, Vitis Vinifera, wild yam, yucca, and Zizyphus jujube.

Grapes skin cuticular wax contains saponins. The saponins discovered in the wines contain ursolic acid, oleanolic acid, ursolic aldehyde, oleanolic aldehyde, hydroxyhopanone, damarenolic acid, mastidienonic acid isomasticadienonic acid. The Vitis Vinifera saponins can be used alone or in association with phenolic compounds such as resveratrol.

### Examples of Serum and Systemic Circulation Concentrations of Saponin Emulsifiers

Serum and systemic circulation concentrations of a saponin effective to result in atherosclerotic plaque regression may vary depending on a number of factors. Influential variables can include, for example, various chemical properties of one saponin, as compared to another. For example different saponins can differ in pKₐ, solubility, molecular weight, etc., and these properties of a particular saponin may affect how a patient metabolizes the saponin, how much of the saponin enters and remains in the systemic circulation of a mammal, and how effectively the saponin emulsifies and dissolves atherosclerotic plaques.

Accordingly, in some embodiments of the present invention, a serum or a systemic circulation concentration of a saponin effective to result in atherosclerotic plaque emulsification and regression may be in a range of from, for instance, about 1 μM to about 10 μM, about 5 μM to about 10 μM, about 10 μM to about 20 μM, about 20 μM to about 30 μM, about 30 μM to about 40 μM, about 40 about μM to about 50 μM, about 50 μM to about 60 μM, about 60 μM to about 70 μM, about 70 μM to about 80 μM, about 80 μM to about 90 μM, about 90 μM to about 100 μM, about 50 μM to about 600 μM, about 50 μM to about 100 μM, about 100 μM to about 300 μM, about 100 μM to about 550 μM, about 150 μM to about 500 μM, about 200 μM to about 450 μM, about 250 μM to about 400 μM, about 300 μM to about 350 μM, about 500 μM to about 600 μM, about 600 μM to about 700 μM, about 700 μM to about 800 μM, about 800 μM to about 900 μM, about 900 μM to about 1 mM, about 1 mM to about 100 mM, about 100 mM to about 200 mM, about 200 mM to about 300 mM, about 300 mM to about 400 mM, about 400 mM to about 500 mM, about 500 mM to about 600 mM, about 600 mM to about 700 mM, about 700 mM to about 800 mM, about 800 mM to about 900 mM, and about 900 mM to about 1 M.

### Examples of Saponin Doses

In some embodiments, a saponin dose effective to result in atherosclerotic plaque emulsification and regression may be, in weight of administered saponin per kilogram of mammal body weight per day (mg/kg/day), in a range of from, for instance, about 1 mg/kg/day to about 10 mg/kg/day, about 10 mg/kg/day to about 20 mg/kg/day, about 20 mg/kg/day to about 30 mg/kg/day, about 30 mg/kg/day to about 40 mg/kg/day, about 40 mg/kg/day to about 50 mg/kg/day, about 50 mg/kg/day to about 60 mg/kg/day, about 60 mg/kg/day to about 100 mg/kg/day, about 100 mg/kg/day to about 125 mg/kg/day, about 125 mg/kg/day to about 150 mg/kg/day, about 150 mg/kg/day to about 175 mg/kg/day, about 175 mg/kg/day to about 200 mg/kg/day, about 200 mg/kg/day to about 225 mg/kg/day, about 225 mg/kg/day to about 250 mg/kg/day, about 250 mg/kg/day to about 275 mg/kg/day, about 275 mg/kg/day to about 300 mg/kg/day, about 300 mg/kg/day to about 325 mg/kg/day, about 325 mg/kg/day to about 350 mg/kg/day, about 350 mg/kg/day to about 375 mg/kg/day, about 375 mg/kg/day to about 400 mg/kg/day, about 400 mg/kg/day to about 425 mg/kg/day, about 425 mg/kg/day to about 450 mg/kg/day, about 450 mg/kg/day to about 475 mg/kg/day, about 475 mg/kg/day to about 500 mg/kg/day, about 500 mg/kg/day to about 550 mg/kg/day, about 550 mg/kg/day to about 600 mg/kg/day, about 600 mg/kg/day to about 650 mg/kg/day, about 650 mg/kg/day to about 700 mg/kg/day, about 700 mg/kg/day to about 750 mg/kg/day, about 750 mg/kg/day to about 800 mg/kg/day, about 800 mg/kg/day to about 850 mg/kg/day, about 850 mg/kg/day to about 900 mg/kg/day, about 900 mg/kg/day to about 950 mg/kg/day, about 950 mg/kg/day to about 1 g/kg/day, about 1 g/kg/day to about 1.25 g/kg/day, about 1.25 g/kg/day to about 1.5 g/kg/day, about 1.5 g/kg/day to about 1.75 g/kg/day, about 1.75 g/kg/day to about 2 g/kg/day, about 2 g/kg/day to about 2.25 g/kg/day, about 2.25 g/kg/day to about 2.5 g/kg/day, about 2.5 g/kg/day to about 2.75 g/kg/day, about 2.750 g/kg/day to about 3 g/kg/day, about 3 g/kg/day to about 4 g/kg/day, about 4 g/kg/day to about 5 g/kg/day, about 5 g/kg/day to about 6 g/kg/day, about 6 g/kg/day to about 7 g/kg/day, about 7 g/kg/day to about 8 g/kg/day, about 8 g/kg/day to about 9 g/kg/day, about 9 g/kg/day to about 10 g/kg/day, about and 10 g/kg/day to about 20 g/kg/day.

### Examples of Detergent Emulsifiers

As used herein, the term “detergent” includes detergents; pharmaceutically acceptable salts, conjugates, hydrates, solvates, derivatives, or polymorphs of detergents; and mixtures thereof. Detergents useful as emulsifiers in certain embodiments described herein include ionic detergents, nonionic detergents, and zwitterionic detergents. Detergents can be used to augment or enhance the effectiveness of other emulsifiers, such as bile acids, terpenes, and/or saponins Detergent can also be used as permeability enhancers, effective to enhance the permeability of membranes or tissue to emulsifiers. Exemplary detergents include the following chemical compounds, sometimes characterized by the following tradenames, and their chemical equivalents and their structural derivatives: reduced TRITON® X-100; reduced TRITON® X-114; TRITON® X-100; NP-40; TRITON® X-114; GENAPOL® X-080; GENAPOL® X-100; C12E8; C12E9; THESIT®; LUBROL® PX; GENAPOL® C-IOO; BRIJ® 35; PLURONIC® F-127®, (laurate); TWEEN® 20 (oleate) and TWEEN® 80; EMPIGEN BB® (n-dodecyl-N,Ndimethylglycine); ZWITTERGENT® 3-08; ZWITTERGENT® 3-10, ZWITTERGENT® 3-12, ZWITTERGENT® 3-14, ZWITTERGENT® 3-16; CHAPS; CHAPSO; ASB-14; ASB-16; DDMAB; DDMAU; EMPIGEN BB® Detergent; and lauryldimethylamine Oxide (LDAO); BATC Cetyltrimethylammonium Bromide (CTAB); Glycholic Acid, Sodium Salt, TOPPS, Molecular Biology Grade Chenodeoxycholic Acid, sodium salt; Molecular Biology Grade Chenodeoxycholic Acid, Free Acid; APO-IO; APO-12; Big CHAP; Big CHAP, deoxy; Cyclohexyl-n-ethyl-β-D-maltoside; ULTROL® Grade; Cyclohexyl-n-hexyl-β-D-maltoside, ULTROL® Grade; Cyclohexyl-n-methyl-β-D-maltoside, ULTROL® Grade; n-Decanoylsucrose; n-Decyl-β-D-maltopyranoside, ULTROL® Grade 252718; n-Decyl-β-D-thiomaltoside, ULTROL® Grade; lauroylsarcosine, Sodium Salt n-Dodecyl Sulfate (SDS); SDS, High Purity; SDS, Molecular Biology Grade; SDS; BRIJ® 35, PROTEIN GRADE® Detergent; C12E6 ELUGENT™ Detergent; GENAPOL® C-100, PROTEIN GRADE® Detergent; GENAPOL® X-80, PROTEIN GRADE® Detergent; GENAPOL® X-IOO, PROTEIN GRADE® Detergent; n-Heptyl-β-D-glucopyranoside; n-Heptyl-β-D-thioglucopyranoside, ULTROL® Grade; n-Hexyl-β-D-glucopyrano side; n-dodecyl-β-D-glucopyranosïde 324355; n-Dodecanoylsucrose 324374; Digitonin; Digitonin, alcohol soluble; MEGA-8, ULTROL® Grade, MEGA-9 ULTROL® Grade, MEGA-10 ULTROL® Grade; n-Nonyl-β-D-glucopyranoside; NP-40, PROTEIN GRADE® Detergent; n-Octanoyl-β-D-glucosylamine (NOGA); π-Octanoylsucrose; n-Octyl-β-D-glucopyranoside; n-Octyl-β-D-glucopyranoside, ULTROL® Grade; n-Octyl-β-D-maltopyranoside; n-Octyl-β-D-thioglycopyranoside, ULTROL® Grade; PLURONIC® F-127, PROTEIN GRADE® Detergent; TRITON® X-100, PROTEIN GRADE® Detergent; TRITON® X-100, Molecular Biology Grade; TRITON® X-100, Hydrogenated; TRITON® X-114, PROTEIN GRADE® Detergent; TWEEN® 20; TWEEN® 20, Molecular Biology Grade Detergent; TWEEN® 20, PROTEIN GRADE® Detergent; TWEEN® 80, PROTEIN GRADE® Detergent; n-Undecyl-B-D-maltoside, ULTROL® Grade Detergent; and lauryldimethylamine oxide.

### Examples of Serum and Systemic Circulation Concentrations of Detergents

Serum and Systemic circulation concentrations of a detergent effective to result in atherosclerotic plaque regression may vary depending on a number of factors. Influential variables can include, for example, various chemical properties of one detergent, as compared to another. For example different detergents can differ in plc, solubility, molecular weight, etc., and these properties of a particular detergent may affect how a patient metabolizes the detergent, how much of the detergent enters and remains in the systemic circulation of a mammal, and how effectively the detergent emulsifies and dissolves atherosclerotic plaques.

Accordingly, in some embodiments of the present invention, a serum or a systemic circulation concentration of a detergent effective to result in atherosclerotic plaques emulsification and regression may be in a range of from, for instance, about 1 μM to about 10 μM, about 5 μM to about 10 μM, about 10 μM to about 20 μM, about 20 μM to about 30 μM, about 30 μM to about 40 μM, about 40 about μM to about 50 μM, about 50 μM to about 60 μM, about 60 μM to about 70 μM, about 70 μM to about 80 μM, about 80 μM to about 90 μM, about 90 μM to about 100 μM, about 50 μM to about 600 μM, about 50 μM to about 100 μM, about 100 μM to about 300 μM, about 100 μM to about 550 μM, about 150 μM to about 500 μM, about 200 μM to about 450 μM, about 250 μM to about 400 μM, about 300 μM to about 350 μM, about 500 μM to about 600 μM, about 600 μM to about 700 μM, about 700 μM to about 800 μM, about 800 μM to about 900 μM, about 900 μM to about 1 mM, about 1 mM to about 100 mM, about 100 mM to about 200 mM, about 200 mM to about 300 mM, about 300 mM to about 400 mM, about 400 mM to about 500 mM, about 500 mM to about 600 mM, about 600 mM to about 700 mM, about 700 mM to about 800 mM, about 800 mM to about 900 mM, and about 900 mM to about 1 M.

### Examples of Detergent Doses

In some embodiments, a detergent dose effective to result in atherosclerotic plaque emulsification and regression may be, in weight of administered detergent per kilogram of mammal body weight per day (mg/kg/day), in a range of from, for instance, about 1 mg/kg/day to about 10 mg/kg/day, about 10 mg/kg/day to about 20 mg/kg/day, about 20 mg/kg/day to about 30 mg/kg/day, about 30 mg/kg/day to about 40 mg/kg/day, about 40 mg/kg/day to about 50 mg/kg/day, about 50 mg/kg/day to about 60 mg/kg/day, about 60 mg/kg/day to about 100 mg/kg/day, about 100 mg/kg/day to about 125 mg/kg/day, about 125 mg/kg/day to about 150 mg/kg/day, about 150 mg/kg/day to about 175 mg/kg/day, about 175 mg/kg/day to about 200 mg/kg/day, about 200 mg/kg/day to about 225 mg/kg/day, about 225 mg/kg/day to about 250 mg/kg/day, about 250 mg/kg/day to about 275 mg/kg/day, about 275 mg/kg/day to about 300 mg/kg/day, about 300 mg/kg/day to about 325 mg/kg/day, about 325 mg/kg/day to about 350 mg/kg/day, about 350 mg/kg/day to about 375 mg/kg/day, about 375 mg/kg/day to about 400 mg/kg/day, about 400 mg/kg/day to about 425 mg/kg/day, about 425 mg/kg/day to about 450 mg/kg/day, about 450 mg/kg/day to about 475 mg/kg/day, about 475 mg/kg/day to about 500 mg/kg/day, about 500 mg/kg/day to about 550 mg/kg/day, about 550 mg/kg/day to about 600 mg/kg/day, about 600 mg/kg/day to about 650 mg/kg/day, about 650 mg/kg/day to about 700 mg/kg/day, about 700 mg/kg/day to about 750 mg/kg/day, about 750 mg/kg/day to about 800 mg/kg/day, about 800 mg/kg/day to about 850 mg/kg/day, about 850 mg/kg/day to about 900 mg/kg/day, about 900 mg/kg/day to about 950 mg/kg/day, about 950 mg/kg/day to about 1 g/kg/day, about 1 g/kg/day to about 1.25 g/kg/day, about 1.25 g/kg/day to about 1.5 g/kg/day, about 1.5 g/kg/day to about 1.75 g/kg/day, about 1.75 g/kg/day to about 2 g/kg/day, about 2 g/kg/day to about 2.25 g/kg/day, about 2.25 g/kg/day to about 2.5 g/kg/day, about 2.5 g/kg/day to about 2.75 g/kg/day, about 2.750 g/kg/day to about 3 g/kg/day, about 3 g/kg/day to about 4 g/kg/day, about 4 g/kg/day to about 5 g/kg/day, about 5 g/kg/day to about 6 g/kg/day, about 6 g/kg/day to about 7 g/kg/day, about 7 g/kg/day to about 8 g/kg/day, about 8 g/kg/day to about 9 g/kg/day, about 9 g/kg/day to about 10 g/kg/day, about and 10 g/kg/day to about 20 g/kg/day.

### Examples of Routes of Administration

Certain embodiments of the present invention comprise routes of administration such as parenteral, transepithelial, transdermal, gavage, oral, oral, sublingual, rectal, vaginal, inhalation, transmucosal, and injection, such as intradermal, subcutaneous, intravenous, and intramuscular injection. In some embodiments, emulsifiers can be perfused directly into the systemic circulation by way of an implantable pump. Regardless of the route of administration, the dosing of emulsifiers will result in achieving sustained levels of an emulsifier in the systemic circulation effective to result in plaque regression.

### Examples of Pharmaceutical Formulations

Certain embodiments of the present invention provide pharmaceutical formulations comprising bile acid, terpene, saponin, and/or detergent atherosclerotic plaque emulsifiers, and at least one of a sustained release delivery system, an absorption enhancing agent, a liposome, a statin, a blood pressure control agent, a lipase, a calcium chelator, a collagenase, a lysyl oxidase agonist, a lysyl oxidase, a lysyl oxidase like protein agonist, a lysyl oxidase like protein, and a pharmaceutically acceptable buffer.

Sustained Release Delivery Systems

In some embodiments, pharmaceutical formulations of the present invention comprise a sustained release delivery system that results in the maintenance of circulating levels of emulsifiers effective to result in plaque regression for extended periods of time, for example, a period of 2 hours or longer. In some embodiments, release is sustained over a period of 24 hours.

In some embodiments, a sustained release delivery system comprises one or more pharmaceutical diluents. Exemplary pharmaceutical diluents include, monosaccharides, disaccharides, polyhydric alcohols, starch, lactose, dextrose, mannitol, sucrose, microcrystalline cellulose, sorbitol, xylitol, fructose, and a combination thereof. In some embodiments, the sustained release delivery system comprises one or more pharmaceutical diluents in an amount of about 5% to about 80% by weight; from about 10% to about 50% by weight; or about 20% by weight of the formulation.

In some embodiments, a sustained release delivery system comprises one or more antiwetting agents, such as a hydrophobic polymer. In certain embodiments, an antiwetting agent is distributed unevenly in the formulation in layers, in pockets, in a coating, or combinations thereof. In certain embodiments, an antiwetting agent is distributed uniformly throughout the formulation. Exemplary hydrophobic polymer antiwetting agents include alkyl celluloses (e.g., C₁₋₆ alkyl celluloses, carboxymethylcellulose), methyl celluloses, ethyl celluloses, propyl celluloses other hydrophobic cellulosic materials or compounds (e.g., cellulose acetate phthalate, hydroxypropylmethylcellulose phthalate), polyvinyl acetate polymers (e.g., polyvinyl acetate phthalate), polymers or copolymers derived from acrylic and/or methacrylic acid esters, zein, waxes (alone or in admixture with fatty alcohols), shellac, hydrogenated vegetable oils, and a combination thereof.

Some embodiments comprise anti-wetting agents in amount of about 0.5% to about 20% by weight of the formulation; in an amount of about 2% to about 10% by weight of the formulation; in an amount of about 3% to about 7% by weight of the formulation; or in an amount of about 5% by weight of the formulation.

In some embodiments, a sustained release delivery system comprises at least one plasticizer, such as triethyl citrate, dibutyl phthalate, propylene glycol, polyethylene glycol, or mixtures of two or more thereof as a coating of the formulation.

In some embodiments, a sustained release delivery system comprises at least one water soluble compound, such as polyvinylpyrrolidone and hydroxypropylmethylcellulose. In certain embodiments, a water soluble compound is distributed unevenly in the formulation in layers, in pockets, as a coating, or combinations thereof. In certain embodiments, a water soluble compound is distributed uniformly throughout the formulation.

In some embodiments, application of a sustained release coating, as described herein, to a formulation may comprise: spraying an aqueous dispersion of the coating onto a core made, for example, by dry or wet granulation of mixed powders of emulsifiers and at least one binding agent; coating an inert bead with emulsifiers and at least one binding agent; and spheronizing mixed powders of emulsifiers and at least one spheronizing agent. Exemplary binding agents include hydroxypropylmethylcelluloses. Exemplary spheronizing agents include microcrystalline celluloses. In some embodiments, the core comprises a tablet made by compressing granules or a powder comprising emulsifiers and/or pharmaceutically acceptable salts or conjugates thereof.

In some embodiments, pharmaceutical formulations comprising emulsifiers and a sustained release delivery system, as described herein, are coated with a sustained release coating, as described herein. In some embodiments, the formulations comprising emulsifiers and a sustained release delivery system, as described herein, are coated with a hydrophobic polymer, as described herein. In some embodiments, the formulations comprising emulsifiers and a sustained release delivery system, as described herein, are coated with an enteric coating. Exemplary enteric coatings include cellulose acetate phthalate, hydroxypropylmethylcellulose phthalate, polyvinylacetate phthalate, methacrylic acid copolymer, shellac, hydroxypropylmethylcellulose succinate, cellulose acetate trimelliate, and a combination thereof.

In some embodiments, the pharmaceutical formulations comprising an emulsifier and a sustained release delivery system, as described herein, are coated with a hydrophobic polymer, as described herein, and further coated with an enteric coating. In any of the embodiments described herein, the formulations comprising emulsifiers and a sustained release delivery system, as described herein, can optionally be coated with a hydrophilic coating which can be applied above or beneath a sustained release film, and/or above or beneath the enteric coating.

Absorption Enhancing Agents

In some embodiments, pharmaceutical formulations of the present invention comprise agents that enhance absorption of bile acid, terpene, saponin, and/or detergent atherosclerotic plaque emulsifiers across, for instance, an intestinal epithelium, a mucosal epithelium, and skin. Absorption enhancing agents include, for example, EDTA, sodium salicylate, sodium caprate, diethyl maleat, N-lauryl-β-D-maltopyranoside, linoleic acid polyoxyethylated, tartaric acid, SDS, Triton X-100, hexylglucoside, hexylmaltoside, heptylglucoside, octylglucoside, octylmaltoside, nonylglucoside, nonylmaltoside, decylglycoside, deceylmaltoside, dodecylmaltoside, tetradecylmaltoside, dodecylglucoside, tridecylmaltoside, as well as mucolytic agents, for example N-acetylcysteine, chitosan, sulfoxides, alcohols, fatty acids and fatty acid esters, polyols, surfactants, terpenes, alkanones, liposomes, ethosomes, cylodextrins, ethanol, glyceryl monoethyl ether, monoglycerides, isopropylmyristate, lauryl alcohol, lauric acid, lauryl lactate, lauryl sulfate, terpinol, menthol, D-limonene, DMSO, polysorbates, N-methylpyrrolidone, polyglycosylated glycerides, Azone®, CPE-215®, NexAct®, SEPA®, and phenyl piperizine. In some embodiments, permeability enhancing agents can also function as emulsifiers.

In some embodiments, bile acid, terpene, saponin, and/or detergent atherosclerotic plaque emulsifiers of the present invention also have properties of permeability enhancing agents, as described herein.

In some embodiments, administration of a pharmaceutical formulation across an epithelium results from at least one of iontophoresis, electroporation, sonophoresis, thermal poration, microneedle treatment, and dermabrasion.

In some embodiments, the pharmaceutical formulation is administered so as to achieve circulating levels of at least 50 μM of the emulsifier within 5 minutes after administration. In some embodiments, administration is performed intravenously. In some embodiments, administration occurs intra-arterially. In some embodiments, levels in a range from about 50 μM to about 600 μM are achieved within 5 minutes of administration. In some embodiments, levels in a range from about 100 μM to about 600 μM are achieved within 5 minutes of administration. In some embodiments, levels in a range from about 100 μM to about 300 μM are achieved within 5 minutes of administration.

Liposomes

Some embodiments of the present invention provide pharmaceutical formulations comprising an active ingredient emulsifier or a combination of active ingredient emulsifiers and unilaminar or multilaminer liposomes having an average diameter in a range of from, for instance, about 100 nm to about 200 nm, about 200 nm to about 300 nm, about 300 nm to about 400 nm, about 400 nm to about 500 nm, about 500 nm to about 600 nm, about 600 nm to about 700 nm, about 700 nm to about 800 nm, about 800 nm to about 900 nm, about 900 nm to about 1.0 micrometer, about 1.0 μm to about 1.25 μm, about 1.250 μm to about 1.5 μm, about 1.5 μm to about 1.75 μm, about 1.75 μm to about 2.0 μm, about 2.0 μm to about 2.25 μm, about 2.25 μm to about 2.5 μm, about 2.5 μm to about 2.75 μm, about 2.75 μm to about 3.0 μm, about 3.0 μm to about 3.25 μm, about 3.25 μm to about 3.5 μm, about 3.5 μm to about 3.75 μm, about 3.75 μm to about 4.0 μm, about 4.0 μm to about 4.5 μm, about 4.5 μm to about 5.0 μm, and about 5.0 μm to about 10.0 μm.

In some embodiments, liposomes comprise lipids and/or phospholipids, such as sphingomyelin, distearoyl-phosphatidylethanolamine (DSPE), distearoyl-phosphatidylcholine (DLPC), phosphatidylcholine (PC), phosphatidylethanolamine (PE), and phosphatidylglycerol (PG).

In some embodiments, a liposomal lipids can be modified with a water soluble polymer, such as a polylactic acid polymer, a polyglycolic acid polymer, a polylactic-polyglycolide copolymer, polyethylene glycol (PEG), polyvinylpyrrolidone, polyacrylamide, polyglycerol, and polyaxozline. In some embodiments, a water soluble polymer comprises an average molecular weight in a range of from, for instance, about 0.1 KDa to about 1.0 KDa, about 1.0 KDa to about 5.0 KDa, about 5.0 KDa to about 25 KDa, about 25 KDa to about 50 KDa, about 50 KDa to about 100 KDa, about 100 KDa to about 250 KDa, about 250 KDa to about 500 KDa, and about 500 KDa to about 1000 KDa. In some embodiments, a covalent bond couples a liposomal lipid to a water soluble polymer.

In some embodiments, liposomal lipids comprising a water soluble polymer comprise an amount of the total liposomal lipids in a range of from, for instance, about 1% to about 10%, about 1% to about 5%, about 10% to about 15%, about 15% to about 20%, about 20% to about 25%, about 25% to about 30%, about 30% to about 40%, about 40% to about 50%, about 50% to about 60%, about 60% to about 70%, about 70% to about 80%, about 80% to about 90%, and about 90% to 100% of the total liposomal lipids.

In some embodiments, liposomes are made by packaging liposomal lipid components with at least one bile acid, terpene, saponin, and/or detergent atherosclerotic plaque emulsifier in water, followed by a lyophilization or an extrusion through, for instance, a membrane comprising pores of a selected average size, such as from about 0.05 μm to about 2.0 1 μM.

In some embodiments, pharmaceutical formulations comprising an active ingredient emulsifier can be percutaneously introduced into the body via percutaneous chemical absorption enhancers such as liposomes, cyclodextrins, and ethosomes. Cyclodextrins comprise a family of cyclic oligosaccharides, composed of 5 or more α-D-glucopyranoside units linked 1->4, as in amylose (a fragment of starch). To date, the largest well-characterized cyclodextrin contains 32 1,4-anhydroglucopyranoside units, but even at least 150-membered cyclic oligosaccharides are known. Typical cyclodextrins contain a number of glucose monomers ranging from six to eight units in a ring that comprise a cone shape. α-cyclodextrin comprises a six membered sugar ring molecule; β-cyclodextrin comprises a seven membered sugar ring molecule; and γ-cyclodextrin comprises an eight membered sugar ring molecule. In some embodiments, a liposome formulation can be administered in an amount that comprises an amount of cyclodextrin in a range of from, for instance, about 1 mg/day to about 10 mg/day, about 10 mg/day to about 20 mg/day, about 20 mg/day to about 30 mg/day, about 30 mg/day to about 40 mg/day, about 40 mg/day to about 50 mg/day, about 50 mg/day to about 60 mg/day, about 60 mg/day to about 70 mg/day, about 70 mg/day to about 80 mg/day, about 80 mg/day to about 90 mg/day, about 90 mg/day to about 100 mg/day, about 100 mg/day to about 150 mg/day, about 150 mg/day to about 300 mg/day, about 300 mg/day to about 500 mg/day, and about 500 mg/day to about 1000 mg/day.

In some embodiments, pharmaceutical formulations comprising an active ingredient emulsifier comprise ethosomes. Ethosomes comprise ultradeformable vesicles having an aqueous core surrounded by a lipid bilayer. Ethosomes comprise at least one amphiphat (such as phosphatidylcholine), which in aqueous solvents self-assembles into a lipid bilayer that closes into a simple lipid vesicle. By including at least one bilayer softening component (such as a biocompatible surfactant or an amphiphile drug) lipid bilayer flexibility and permeability are greatly increased. The resulting, flexibility and permeability optimized, ethosome can therefore adapt its shape easily and rapidly, by adjusting local concentration of each bilayer component to the local stress experienced by the bilayer. In its basic organization, broadly similar to a liposome, the ethosome differs from more conventional vesicle primarily by its “softer,” more deformable and adjustable membrane. A consequence an ethosome's strong bilayer deformability is the increased ethosome affinity to bind and retain water. An ultradeformable and highly hydrophilic vesicle always tends to avoid dehydration. For example, an ethosome vesicle applied on an open biological surface, such as non-occluded skin, tends to penetrate its barriers and migrate into the water-rich deeper strata to secure hydration. Barrier penetration by ethosomes involves reversible bilayer deformation, without compromising either the vesicle integrity or the barrier properties for the underlying hydration affinity and gradient to remain in place. Being too large to diffuse through the skin, the ethosome needs to find its own route through the organ. The ethosome vesicles use in drug delivery consequently relies on the carrier's ability to widen and overcome the hydrophilic pores in the skin. A concomitant gradual drug agent release from the ethosome allows drug molecules to diffuse and bind to target. Drug transport by an ethosome to an intra-cellular action site may also involve ethosome carrier lipid bilayer fusion with a cell membrane, or active ethosome uptake by the cell by, e.g. endocytosis.

Ethosomes provide for non-invasive delivery of therapeutic molecules across open biological barriers. Ethosome vesicles can transport across mammalian skin, for example, molecules that are too big to diffuse through skin barriers. Other applications include the transport of small molecule drugs which have certain physicochemical properties which would otherwise prevent them from diffusing across a skin barrier. Another characteristic of certain ethosomes is an ability to deliver active drug agents to peripheral, subcutaneous tissue. This ability relies on minimization of the carrier-associated drug clearance through a cutaneous blood vessels plexus in which non-fenestrated blood capillary walls in the skin that, together with the tight junctions between endothelial cells, preclude vesicles getting directly into blood. Ethosome vesicles are prepared in a similar manner as liposomes, except that no separation of the vesicle-associated and free drug is required. Examples include sonicating, extrusion, low shear rates mixing (multilamellar liposomes), or high high-shear homogenizations unilamellar liposomes) of the crude vesicle suspension.

In some embodiments, pharmaceutical formulations comprising an active ingredient emulsifier comprise ethosomes in a range of weight:weight or weight:volume percentages of from about 1% to about 5%, about 1% to about 10%, about 10% to about 15%, about 15% to about 20%, about 20% to about 25%, about 25% to about 30%, about 30% to about 40%, about 40% to about 50%, about 50% to about 60%, and about 60% to about 70%.

In some embodiments, liposomes are subjected to both lyophilization and extrusion. Some embodiments provide inhalation pharmaceutical formulations comprising liposomes suitable for administration with an inhaler, such as a metered dose inhaler, a dry powder inhaler, and a jet nebulizer. Some embodiments provide pharmaceutical formulations comprising liposomes suitable for administration by injection. Some embodiments provide topical pharmaceutical formulations comprising liposomes, such as creams, lotions, emulsions, pastes, and ointments, which can transdermally deliver a lipo-dissolving compound, such as a bile acid, terpene, saponin, and/or detergent compound. In some embodiments, formulations comprising liposomes include compounds which assist fat metabolism, such as phosphatidylcholine and/or L-carnitine.

In some embodiments, a liposome formulation can be administered in an amount that comprises an amount of liposome in a range of weight:weight or weight:volume percentages of from about 1% to about 5%, about 1% to about 10%, about 10% to about 15%, about 15% to about 20%, about 20% to about 25%, about 25% to about 30%, about 30% to about 40%, about 40% to about 50%, about 50% to about 60%, and about 60% to about 70%. The statin and emulsifier can be administered concurrently, or sequentially. In some embodiments, the statin and emulsifier can be provided in the same pharmaceutical composition, either as a mixture or in sub-compartments of a single dosage form such as a pill, capsule, injectable, or any other suitable form for administration

Statins

In some embodiments, a method of treating a patient having atherosclerotic plaques, or at risk of having atherosclerotic plaques due to, for instance, a family history or lifestyle predisposition toward plaque development, comprises treatment with an emulsifier as described above, in combination with agents effective to lower cholesterol. For example, a class of compounds known as “statins” are effective to lower cholesterol. Statins are inhibitors of HMG-CoA reductase, the rate limiting enzyme in the synthesis of mevalonate, a key intermediate in the synthesis of cholesterol, from acetyl-CoA.

A variety of natural and synthetic statins are known. These include, for example and without being limiting, atorvastatin, cerivastatin, fluvastatin, lovastatin, mevastatin, pitavastatin, pravastatin, rosuvastatin, and simvastatin. In some embodiments, a method of treating atherosclerosis, effective to result in a reduction in plaque volume, comprises treatment with an emulsifier as described above in combination with a statin.

Doses of statins, administered in combination with at least one active ingredient emulsifier of the present invention, effective to result in regression of an atherosclerotic plaque may vary depending on a number of factors. Influential variables can include, for example, various chemical properties of one statin, as compared to another. For example different statins can differ in pKₐ, solubility, molecular weight, etc., and these properties of a particular statin may affect how a patient metabolizes the statin, how much of the statin enters and remains in the systemic circulation of a mammal, and how effectively the statin emulsifies and dissolves atherosclerotic plaques.

Accordingly, in some embodiments, a statin dose comprises an amount of statin in a range of from, for instance, about 1 mg/day to about 10 mg/day, about 10 mg/day to about 20 mg/day, about 20 mg/day to about 30 mg/day, about 30 mg/day to about 40 mg/day, about 40 mg/day to about 50 mg/day, about 50 mg/day to about 60 mg/day, about 60 mg/day to about 70 mg/day, about 70 mg/day to about 80 mg/day, about 80 mg/day to about 90 mg/day, about 90 mg/day to about 100 mg/day, about 100 mg/day to about 150 mg/day, about 150 mg/day to about 300 mg/day, about 300 mg/day to about 600 mg/day, and about 500 mg/day to about 1000 mg/day. The statin and emulsifier can be administered concurrently, or sequentially. In some embodiments, the statin and emulsifier can be provided in the same pharmaceutical composition, either as a mixture or in sub-compartments of a single dosage form such as a pill, capsule, injectable, or any other suitable form for administration.

Blood Pressure Control Agents

In some embodiments, emulsifiers can be administered in combination with a an agent effective to control blood pressure. For example, in some embodiments emulsifiers are provided simultaneously, or sequentially, with a statin and a compound like amlodipine.

Lipases

Lipases, a subclass of esterases, comprise water-soluble enzymes that catalyze hydrolysis of ester bonds in water-insoluble lipids. Several distinct lipase enzymes are found in nature, and most lipases act at a specific position on the glycerol backbone of a lipid substrate. In addition, most lipases comprise an alpha/beta hydrolase fold and employ a chymotrypsin-like lipid hydrolysis mechanism involving a serine nucleophile, an acid residue (usually aspartic acid), and a histidine. Several lipases hydrolyze lipidic components of atherosclerotic plaques.

In some embodiments, emulsifiers as described above can be administered in combination with at least one lipase. Exemplary lipases include pancreatic lipase (HPL), hepatic lipase (HL), endothelial lipase, lipoprotein lipase (LPL), lysosomal lipase (LIPA, and also known as acid cholesteryl ester hydrolase), hepatic lipase (LIPC), hormone-sensitive lipase, pancreatic lipase related protein 1 (PLRP1), pancreatic lipase related protein 2 (PLRP2), phospholipases, lipase H (LIPH), lipase I (LIPI), lipase J (LIPJ), lipase K (LIPK), lipase M (LIPM), lipase N (LIPN), monoglyceride lipase (MGLL), diacylglyceride lipase alpha (DAGLA), diacylglyceride lipase beta (DAGLB), and carboxyl ester lipase (CEL).

Doses of lipases, administered in combination with at least one active ingredient emulsifier of the present invention, effective to result in regression of an atherosclerotic plaque may vary depending on a number of factors. Influential variables can include, for example, various chemical properties of one lipase, as compared to another. For example different lipases can differ in pKₐ, solubility, molecular weight, etc., and these properties of a particular lipase may affect how a patient metabolizes the lipase, how much of the lipase enters and remains in the systemic circulation of a mammal, and how effectively the lipase emulsifies and dissolves atherosclerotic plaques.

Accordingly, in some embodiments, a lipase dose comprises an amount of lipase in a range of from, for instance, about 1 mg/day to about 10 mg/day, about 10 mg/day to about 20 mg/day, about 20 mg/day to about 30 mg/day, about 30 mg/day to about 40 mg/day, about 40 mg/day to about 50 mg/day, about 50 mg/day to about 60 mg/day, about 60 mg/day to about 70 mg/day, about 70 mg/day to about 80 mg/day, about 80 mg/day to about 90 mg/day, about 90 mg/day to about 100 mg/day, about 100 mg/day to about 150 mg/day, about 150 mg/day to about 300 mg/day, about 300 mg/day to about 600 mg/day, and about 500 mg/day to about 1000 mg/day. The lipase and emulsifier can be administered concurrently, or sequentially. In some embodiments, the lipase and emulsifier can be provided in the same pharmaceutical composition, either as a mixture or in sub-compartments of a single dosage form such as a pill, capsule, injectable, or any other suitable form for administration.

Calcium Chelators

Calcium deposits are frequently present in atherosclerotic plaques. In some embodiments, emulsifiers as described above can be administered in combination with at least one calcium chelating agent. Exemplary calcium chelators include 1,2-Bis(2-aminophenoxy)ethane-N,N,N′,N′-tetraacetic acid, BAPTA-AM, Ethylene glycol-bis(2-aminoethylether)-N,N,N′,N′-tetraacetic acid, Ethylene glycol-bis(β-aminoethyl ether)-N,N,N′,N′-tetraacetic acid tetrasodium salt, and ethylenediaminetetraacetic acid disodium salt dihydrate reagent grade, ˜99% (titration).

Doses of calcium chelators, administered in combination with at least one active ingredient emulsifier of the present invention, effective to result in regression of an atherosclerotic plaque may vary depending on a number of factors. Influential variables can include, for example, various chemical properties of one calcium chelator, as compared to another. For example different calcium chelators can differ in pKₐ, solubility, molecular weight, etc., and these properties of a particular calcium chelator may affect how a patient metabolizes the calcium chelator, how much of the calcium chelator enters and remains in the systemic circulation of a mammal, and how effectively the calcium chelator emulsifies and dissolves atherosclerotic plaques.

Accordingly, in some embodiments, a calcium chelator dose comprises an amount of calcium chelator in a range of from, for instance, about 1 mg/day to about 10 mg/day, about 10 mg/day to about 20 mg/day, about 20 mg/day to about 30 mg/day, about 30 mg/day to about 40 mg/day, about 40 mg/day to about 50 mg/day, about 50 mg/day to about 60 mg/day, about 60 mg/day to about 70 mg/day, about 70 mg/day to about 80 mg/day, about 80 mg/day to about 90 mg/day, about 90 mg/day to about 100 mg/day, about 100 mg/day to about 150 mg/day, about 150 mg/day to about 300 mg/day, about 300 mg/day to about 600 mg/day, and about 500 mg/day to about 1000 mg/day. The calcium chelator and emulsifier can be administered concurrently, or sequentially. In some embodiments, the calcium chelator and emulsifier can be provided in the same pharmaceutical composition, either as a mixture or in sub-compartments of a single dosage form such as a pill, capsule, injectable, or any other suitable form for administration.

Collagenases

Collagen is a component of the fibrous cap present in many atherosclerotic plaques. Collagenases comprise a group of enzymes, made by a variety of microorganisms and animal cells, that break down collagens. In some embodiments, emulsifiers as described above can be administered in combination with at least one collagenase. Exemplary collagenases include both crude and purified Clostridium histolyticum collagenases as well as mammalian matrix metalloproteinases (MMPs), MMP 1, MMP 2, MMP 8, MMP 8, MMP 13, MMP 14, and MMP 18.

Doses of collagenases, administered in combination with at least one active ingredient emulsifier of the present invention, effective to result in regression of an atherosclerotic plaque may vary depending on a number of factors. Influential variables can include, for example, various chemical properties of one collagenase, as compared to another. For example different collagenases can differ in pKₐ, solubility, molecular weight, etc., and these properties of a particular collagenase may affect how a patient metabolizes the collagenase, how much of the collagenase enters and remains in the systemic circulation of a mammal, and how effectively the collagenase emulsifies and dissolves atherosclerotic plaques.

Accordingly, in some embodiments, a collagenase dose comprises an amount of collagenase in a range of from, for instance, about 1 mg/day to about 10 mg/day, about 10 mg/day to about 20 mg/day, about 20 mg/day to about 30 mg/day, about 30 mg/day to about 40 mg/day, about 40 mg/day to about 50 mg/day, about 50 mg/day to about 60 mg/day, about 60 mg/day to about 70 mg/day, about 70 mg/day to about 80 mg/day, about 80 mg/day to about 90 mg/day, about 90 mg/day to about 100 mg/day, about 100 mg/day to about 150 mg/day, about 150 mg/day to about 300 mg/day, about 300 mg/day to about 600 mg/day, and about 500 mg/day to about 1000 mg/day. The collagenase and emulsifier can be administered concurrently, or sequentially. In some embodiments, the collagenase and emulsifier can be provided in the same pharmaceutical composition, either as a mixture or in sub-compartments of a single dosage form such as a pill, capsule, injectable, or any other suitable form for administration.

Hematoporphyrins

Hematoporphyrins are selectively absorbed into atherosclerotic plaques, with little or no absorption into healthy areas of the arterial wall. In some embodiments, emulsifiers as described above can be administered with hematoporphyrins, effective to target the emulsifier to atherosclerotic plaques. In some embodiments, a hematoporphyrin dose comprises an amount of hematoporphyrin in a range of from, for instance, about 1 mg/day to about 10 mg/day, about 10 mg/day to about 20 mg/day, about 20 mg/day to about 30 mg/day, about 30 mg/day to about 40 mg/day, about 40 mg/day to about 50 mg/day, about 50 mg/day to about 60 mg/day, about 60 mg/day to about 70 mg/day, about 70 mg/day to about 80 mg/day, about 80 mg/day to about 90 mg/day, about 90 mg/day to about 100 mg/day, about 100 mg/day to about 150 mg/day, about 150 mg/day to about 300 mg/day, about 300 mg/day to about 600 mg/day, and about 500 mg/day to about 1000 mg/day. The hematoporphyrin and emulsifier can be administered concurrently, or sequentially. In some embodiments, the hematoporphyrin and emulsifier can be provided in the same pharmaceutical composition, either as a mixture or in sub-compartments of a single dosage form such as a pill, capsule, injectable, or any other suitable form for administration.

Lysyl Oxidase

Lysyl oxidase and lysyl oxidase like proteins catalyze deamination of peptidyl lysine and hydroxylysine residues in collagens and peptidyl lysine residues in elastin. The resulting peptidyl aldehydes undergo oxidation reactions to form lysine-derived covalent cross-links required for structural integrity of collagen and elastin extracellular matrix components. By such activities, lysyl oxidases and lysyl oxidase like proteins can be considered as targets for inducing elastogenesis in a variety of contexts. Lysyl oxidase and lysyl oxidase like proteins are synthesized as proenzymes, secreted into the extracellular environment, and processed by proteolytic cleavage into an enzymatically active peptide and a propeptide. Its stimulation by a dill extract is correlated with increased elastin detection, suggesting an increase in elastogenesis efficiency.

Disruptions in lysyl oxidase expression and activity have been linked to atherosclerosis. In some embodiments, lysyl oxidase and lysyl oxidase like protein agonists, which upregulate lysyl oxidase expression, lysyl oxidase like protein activity, or combinations thereof, can be administered in combination with emulsifiers as described above. Exemplary agonists of lysyl oxidase include transformation growth factor-beta, granulocyte macrophage colony stimulating factor, suramin, dill, dill extract, Anethum graveolens extract, Lys'lastin V, lysyl oxidase, and lysyl oxidase like proteins.

Doses of lysyl oxidase agonists, administered in combination with at least one active ingredient emulsifier of the present invention, effective to result in regression of an atherosclerotic plaque may vary depending on a number of factors. Influential variables can include, for example, various chemical properties of one lysyl oxidase agonist, as compared to another. For example different lysyl oxidase agonists can differ in pKₐ, solubility, molecular weight, etc., and these properties of a particular lysyl oxidase agonist may affect how a patient metabolizes the lysyl oxidase agonist, how much of the lysyl oxidase agonist enters and remains in the systemic circulation of a mammal, and how effectively the lysyl oxidase agonist emulsifies and dissolves atherosclerotic plaques.

Accordingly, in some embodiments, a lysyl oxidase agonist dose comprises an amount of lysyl oxidase agonist in a range of from, for instance, about 1 mg/day to about 10 mg/day, about 10 mg/day to about 20 mg/day, about 20 mg/day to about 30 mg/day, about 30 mg/day to about 40 mg/day, about 40 mg/day to about 50 mg/day, about 50 mg/day to about 60 mg/day, about 60 mg/day to about 70 mg/day, about 70 mg/day to about 80 mg/day, about 80 mg/day to about 90 mg/day, about 90 mg/day to about 100 mg/day, about 100 mg/day to about 150 mg/day, about 150 mg/day to about 300 mg/day, about 300 mg/day to about 600 mg/day, and about 500 mg/day to about 1000 mg/day. The lysyl oxidase agonist and emulsifier can be administered concurrently, or sequentially. In some embodiments, the lysyl oxidase agonist and emulsifier can be provided in the same pharmaceutical composition, either as a mixture or in sub-compartments of a single dosage form such as a pill, capsule, injectable, or any other suitable form for administration.

### Examples of Stents

Emulsifiers, as well as other therapeutic compounds, for example, statins, can be administered by way of a stent. In some embodiments, after an angioplasty procedure, a stent comprising at least one emulsifier as described above, can be placed in a vessel at the site of the angioplasty. The stent is configured to release the emulsifiers in a sustained fashion, such that a local concentration that is effective to dissolve plaques is achieved. The stent can be loaded with one or more emulsifiers, and/or additional therapeutic compounds, and configured to release the therapeutic ingredients over an extended period of time. In some embodiments, the local concentration of emulsifier provided by the stent can be greater than 50 μm. In some embodiments, the local concentration of emulsifier can be in a range from about 50 μM to about 600 μM. In some embodiments, the local concentration of the emulsifier can range from about 100 μM to about 300 μM. Emulsifier eluting stents can be of a balloon-expandable design, or self-expanding. The stent can also include additional agents effective to dissolve plaque, for example, ionic detergents, nonionic detergents, and zwitterionic detergents. An exemplary list of detergents is provided in International Application PCT/US2007/001214, the entire contents of which are incorporated by reference herein.

In some embodiments, a stent comprises enzymes that digest plaque components (e.g., the fibrous cap), such as proteases, including collagenase, pronase, proteinase K, trypsin, and chymotrypsin. In some embodiments, proteases comprise, without being limiting, serine proteases, threonine proteases, cysteine proteases, aspartic acid proteases, metalloproteases, and glutamic acid proteases. As such, the enzymes listed are understood to be merely exemplary and not exhaustive of the enzymes that can be included in a stent configured for sustained release of emulsifiers. Proteolytic enzymes that are effective to dissolve blood clots, can also be useful in embodiments of stents that release active ingredient emulsifiers and combinations of such emulsifiers, in order to prevent, reduce, or limit, the risk of forming a thrombus at or near a site where the stent is placed in the patient. A stent can also include other therapeutic agents such as anti-inflammatory compounds, or compounds that are effective to promote healing of the vessel.

### EXPERIMENTAL EXAMPLES

### Protocol 1

Protocol 1 provides an in vitro assay for determining the specificity and/or effectiveness with which a bile acid, terpene, saponin, and/or detergent emulsifier, or a combination of such emulsifiers, or a pharmaceutical formulation comprising such an emulsifier or emulsifier combination, emulsifies and dissolves atherosclerotic plaque components. In protocol 1, test and control solutions are prepared. Test solution comprises at least one bile acid, terpene, saponin, and/or detergent emulsifier at a weight:volume ratio (w:v) in, for example, a range of from 1 ng/ml to 10 ng/ml, 10 ng/ml to 100 ng/ml, 100 ng/ml to 500 ng/ml, 500 ng/ml to 1 μg/ml, 1 μg/ml to 10 μg/ml, 10 μg/ml to 100 μg/ml, 100 μg/ml to 500 μg/ml, 500 μg/ml to 1 mg/ml, 1 mg/ml to 10 mg/ml, 10 mg/ml to 100 mg/ml, 100 mg/ml to 500 mg/ml, or 500 mg/ml to 1 g/ml. Control solution differs from test solution by lacking at least one emulsifier present in the test solution. When the test and/or control solutions comprise more than one emulsifier, the w:v ratio of each emulsifier in solution can be the same or different. The test and control solutions can comprise aqueous solutions, organic solvents, and combinations thereof.

Equal amounts of solid aggregate comprising at least one atherosclerotic plaque component are independently combined with equal volumes of test and control solution. Exemplary amounts of aggregate include about 1 ng, about 10 ng, about 100 ng, about 500 ng, about 1 μg, about 10 μg, about 100 μg, about 500 μg, about 1 mg, about 10 mg, about 100 mg, about 500 mg, and about 1 g. Exemplary volumes of test and control solutions include about 1 μl, about 10 μl, about 100 ml, about 1 ml, about 10 ml, about 100 ml, and about 1 l.

The aggregate and test and control solutions are incubated at about 15° C., about 20° C., about 25° C., about 30° C., about 35° C., about 37° C., about 40° C., about 45° C., or about 50° C. for a period of, for example, 1 minute, 5 minutes, 10 minutes, 15 minutes, 30 minutes, 45 minutes, 1 hour, 2 hours, 4 hours, 8 hours, 16 hours, 1 day, 2 days, 3 days, 4 days, 5 days, 6 days, 1 week, or 2 weeks. During the incubation period the test and control solutions can be subjected to the same amount of continuous or intermittent agitation, such as stirring, rocking, and/or shaking. After the incubation period, the amount of aggregate dissolved in each test and control solution is determined and compared, providing the effectiveness with which the tested emulsifier, combination of emulsifiers, or pharmaceutical formulation emulsifies and dissolves the aggregated plaque component.

Methods for determining and/or quantifying the amount of emulsified plaque components in a solution are known and include, without limitation, enzyme linked immunoassay (ELISA), high performance liquid chromatography, fast protein liquid chromatography, gas chromatography, thin layer paper chromatography, mass spectrometry, volume displacement. In addition, methods for determining and/or quantifying the amount of non-emulsified plaque components (e.g. solid aggregate remaining out of solution), such as weighing and visual inspection, are also known.

The specificity with which a bile acid, terpene, saponin, and/or detergent atherosclerotic plaque emulsifier, or a pharmaceutical formulation comprising such emulsifiers, emulsifies atherosclerotic plaque components can be determined by including in this protocol one or more additional test solutions, independently combined with an aggregated lipid not found in atherosclerotic plaques, such as lard. Such additional test and control solutions are processed in the manner described herein, and provide the specificity with which the tested emulsifier, combination of emulsifiers, or pharmaceutical formulation emulsifies and dissolves aggregated plaque components. For instance, a significantly plaque-specific emulsifier, combination of emulsifiers, or pharmaceutical formulation will emulsify and dissolve a substantially greater amount of a solid lipid aggregate comprising plaque lipids as compared to the amount of solid lipid aggregate comprising non-plaque lipids it emulsifies and dissolves. A general emulsifier emulsifies and dissolves substantially similar amounts of solid lipid aggregates comprised of plaque and non-plaque lipids.

### Protocol 2

Protocol 2 provides an ex vivo assay for determining the effectiveness with which a bile acid, terpene, saponin, and/or detergent atherosclerotic plaque emulsifier, or a pharmaceutical formulation comprising such an emulsifier or combination of emulsifiers, emulsifies and dissolves atherosclerotic plaques. In protocol 2, ex vivo samples of a mammalian artery comprising atherosclerotic plaques are independently combined with test and control solutions prepared as described in protocol 1, and incubated as described in protocol 1. A size of the plaques, e.g., area, volume, or thickness, is measured before and after incubation with the test or control solutions. Comparison of these size measurements provides the effectiveness with which the tested emulsifier, combination of emulsifiers, or pharmaceutical formulation emulsifies and dissolves atherosclerotic plaques.

### Protocol 3

Protocol 3 provides an in vivo assay for determining the effectiveness with which a bile acid, terpene, saponin, and/or detergent atherosclerotic plaque emulsifier, or a pharmaceutical formulation comprising such an emulsifier or combination of emulsifiers, emulsifies and dissolves atherosclerotic plaques.

In weeks 1 to 8 of protocol 3, four groups of substantially genetically identical mice, Groups A, B, C, and D, each comprising four to twelve animals, are housed in humidity and temperature controlled conditions and fed a high fat and/or high cholesterol rodent chow, such as Picolab Rodent Chow 20 (5053) pellets containing 0.5% (w/w) cholesterol, to promote atherosclerotic plaque formation.

Starting at week 9, Group A mice are fed the high fat and/or high cholesterol rodent chow supplemented with a first emulsifier, such as the bile acid hyodeoxycholic acid (HDCA); Group B mice are fed the high fat and/or high cholesterol rodent chow supplemented with a second emulsifier, such as the terpene emulsifier D-limonene or metabolite thereof; Group C mice are fed the high fat and/or high cholesterol rodent chow supplemented with a combination of the first and second emulsifiers; and Group D mice are fed the high fat and/or high cholesterol rodent chow. Alternatively, Group A mice are fed the high fat and/or high cholesterol rodent chow and administered the first emulsifier by injection, suppository, topical formulation, etc.; Group B mice are fed the high fat and/or high cholesterol rodent chow and administered the second emulsifier by injection, suppository, topical formulation, etc.; Group C mice are fed the high fat and/or high cholesterol rodent chow and administered the combination of the first and second emulsifiers by injection, suppository, topical formulation, etc. The doses of first and second emulsifiers administered to the animals are as described herein.

Starting at week 10, systemic circulation levels of cholesterol and the first and second emulsifiers and/or their precursors, derivatives, metabolites, etc., such as HDCA and D-limonen or S-perillic acid, can be measured in the systemic circulations of the mice of Groups A, B, C, and D. Assays for determining levels of cholesterol and emulsifiers in blood are known in the art, and include, without limitation, ELISA, high performance liquid chromatography, fast protein liquid chromatography, gas chromatography, thin layer paper chromatography, and mass spectrometry. The concentrations of first and second emulsifiers achieved the systemic circulation in mice of groups A, B, and C are as described herein.

At the end of week 25, animals in Groups A, B, C, and D are sacrificed. After sacrifice, the blood of each mouse in Groups A, B, C, and D is removed, and the circulatory system is perfused with phosphate-buffered saline by intraventricular injection. The heart, containing the aortic root, is fixed in phosphate-buffered formalin and processed, by known methods, for aortic root quantitative assessment of atherosclerotic plaque size, e.g., an area, volume, and/or thickness, for example, by the assay described in Dansky et al., 1999. Arterioscler. Thromb. Vasc. Biol. 19:1960-1968, the entire contents of which are hereby incorporated by reference in their entirety. A comparison of atherosclerotic plaque size measurements between the animals of Groups A, B, C, and D provides the effectiveness with which the tested emulsifier and combination of emulsifiers promotes plaque regression.

### Experiment 1

In vitro experiments were performed to assess the specificity and effectiveness with which S-perillic acid, a metabolite of D-limonene, emulsifies and dissolves aggregates comprising lipidic atherosclerotic plaque components. In these experiments, 1.0 g of S-perillic acid was dissolved in 50 ml of an aqueous solution comprising 50 mM HEPES, pH 7.3, and distributed into 10 ml aliquots. 0.11 g each of aggregated cholesteryl oleate, cholesteryl palmitate, cholesterol crystals, and lard was placed in independent S-perillic acid/HEPES aliquots, creating test samples. Each test sample was incubated at room temperature for 84 hours, without stirring for the initial 72 hours and then with continuous stirring for 12 hours.

A control solution comprising 50 mM HEPES, pH 7.3, was distributed into 10 ml aliquots, and 0.11 g each of aggregated cholesteryl oleate, cholesteryl palmitate, cholesterol crystals, and lard was placed in independent HEPES aliquots, creating control samples. The control samples were processed in the same manner as the test samples: i.e., incubated at room temperature without stirring for the initial 72 hours and with continuous stirring for 12 hours.

After incubation, 90-95% of each of the aggregated cholesteryl oleate, cholesteryl palmitate, and cholesterol crystals of the test samples had dissolved. In contrast, the aggregated cholesteryl oleate, cholesteryl palmitate, and cholesterol crystals of the control samples remained insoluble. In addition, aggregated lard remained insoluble in both the test and control samples.

These experiments demonstrate that S-perillic acid is soluble in an aqueous solution comprising 50 mM HEPES, pH 7.3, and has a property of being an effective and specific emulsifier of aggregated atherosclerotic plaque lipids. It is believed that these results indicate a substantial number of D-limonene metabolites are likely significantly specific emulsifiers of atherosclerotic plaque lipids that provide pharmacological advantages over general emulsifiers, in terms of efficacy, safety and tolerability, and are effective to dissolve aggregated, insoluble cholesterol components of atherosclerotic plaques in vivo.

### Experiment 2

In vitro experiments were performed to assess the specificity with which S-perillyl alcohol, a metabolite of terpene emulsifier D-limonene, emulsifies and dissolves aggregates comprising lipidic atherosclerotic plaque components. In these experiments, 1.0 g of a 96% solution of S-perillyl alcohol was mixed with 1 ml of an aqueous solution comprising 50 mM HEPES, pH 7.3, and distributed into 0.5 ml or 1.5 ml aliquots. 0.03 g each of aggregated cholesteryl oleate and cholesteryl palmitate was placed in independent S-perillyl alcohol/HEPES 0.5 ml aliquots, and 0.03 g of cholesterol crystals was placed in a 1.5 ml aliquot of S-perillyl alcohol/HEPES, creating test samples. Each test sample was incubated at room temperature for 2 hours, with intermittent shaking.

A control solution comprising 50 mM HEPES, pH 7.3, was distributed into 1.5 ml aliquots, and 0.03 g each of aggregated cholesteryl oleate, cholesteryl palmitate, and cholesterol crystals, was placed in independent HEPES aliquots, creating control samples. The control samples were processed in the same manner as the test samples: i.e., incubated at room temperature for 2 hours, with intermittent shaking.

After incubation, 90-95% of each of the aggregated cholesteryl oleate, cholesteryl palmitate, and cholesterol crystals of the test samples had dissolved. In contrast, the aggregated cholesteryl oleate, cholesteryl palmitate, and cholesterol crystals of the control samples remained insoluble. In addition, the aggregated cholesteryl oleate, cholesteryl palmitate, and cholesterol crystals of the control samples remained insoluble after an extended incubation of 36 hours.

These experiments demonstrate that S-perillyl alcohol, a metabolite of the terpene emulsifier D-limonene, has a property of being an effective emulsifier of aggregated atherosclerotic plaque lipids. It is believed that these results indicate a substantial number of D-limonene metabolites are effective emulsifiers of atherosclerotic plaques in vivo.

### Experiment 3.1

In vitro experiments were performed to assess the effectiveness with which HDCA emulsifies and dissolves aggregates comprising lipidic atherosclerotic plaque components. In these experiments, 10.0 g of HDCA was dissolved in 40 ml of 96% ethanol, diluted with 40 ml of water, and distributed into 40 ml aliquots. 0.20 g each of aggregated cholesteryl oleate and cholesterol crystals was placed in independent HDCA/ethanol aliquots, creating test samples. Each test sample was incubated at room temperature for six hours with continuous stirring.

A control solution comprising 40 ml 96% ethanol diluted with 40 ml water was distributed into 40 ml aliquots, and 0.20 g each of aggregated cholesteryl oleate and cholesterol crystals were placed in independent ethanol aliquots, creating control samples. The control samples were processed in the same manner as the test samples: i.e., incubated at room temperature with continuous stirring for 6 hours.

After incubation, approximately 95% of the cholesterol crystals of the test sample had dissolved. In contrast, the aggregated cholesteryl oleate of both the test and control samples and the cholesterol crystals of the control samples remained insoluble. These experiments demonstrate that HDCA has a property of being a effective emulsifier of aggregated atherosclerotic plaque lipids.

### Experiment 3.2

Further in vitro experiments were performed to assess the effectiveness with which HDCA emulsifies and dissolves cholesteryl oleate aggregates. In these experiments, 10.0 g of HDCA was dissolved in 40 ml of 96% ethanol, and distributed into a 40 ml aliquot. 0.20 g of aggregated cholesteryl oleate was placed in the HDCA/ethanol test aliquot, creating a test sample that was incubated at about 37° C. with shaking for about five minutes.

A control solution comprising 40 ml 96% ethanol was distributed into a 40 ml aliquot, and 0.20 g of aggregated cholesteryl oleate was placed into the ethanol aliquot to create a control sample. The control sample was processed in the same manner as the test samples: i.e., incubated at about 37° C. with shaking for five minutes.

After incubation, approximately 70% of the aggregated cholesteryl oleate of the test sample had dissolved. In contrast, the aggregated cholesteryl oleate of the control sample remained insoluble.

These experiments demonstrate that HDCA has a property of being a significantly effective emulsifier of aggregated atherosclerotic plaque lipids. It is believed that these results indicate a substantial number of bile acids are effective emulsifiers of atherosclerotic plaques in vivo.

### Experiment 4

In vitro experiments were performed to determine the effectiveness with which a combination of HDCA and D-limonene emulsifies and dissolves aggregates comprising lipidic atherosclerotic plaque components. In these experiments, 7.5 g of HDCA and 7.5 g of D-limonene were combined with 75 ml of a solution comprising 70% isopropanol and 30% water and distributed into 25 ml aliquots. 0.11 g each of aggregated cholesteryl oleate and cholesterol crystals were placed in independent HDCA-D-limonene aliquots, creating test samples. Each test sample was incubated at room temperature for fifteen minutes with continuous stirring.

A control solution comprising 70% isopropanol and 30% water was distributed into 25 ml aliquots, and 0.11 g each of aggregated cholesteryl oleate and cholesterol crystals were placed in independent control aliquots, creating control samples. The control samples were processed in the same manner as the test samples: i.e., incubated at room temperature for 15 minutes with continuous stirring.

After incubation, 100% of each of the aggregated cholesteryl oleate and cholesterol crystals of the test samples had dissolved. In contrast, the aggregated cholesteryl oleate, cholesteryl palmitate, and cholesterol crystals of the control samples remained insoluble.

These experiments demonstrate that combinations of HDCA and D-limonene have a property of being a significantly effective emulsifier of aggregated atherosclerotic plaque lipids. It is believed that these results indicate a substantial number of bile acid and terpene combinations are effective emulsifiers of atherosclerotic plaques in vivo.

### Experiment 5

In vitro experiments were performed to determine the effectiveness with which a combination of DCA and D-limonene emulsifies and dissolves aggregates comprising lipidic atherosclerotic plaque components. In these experiments, 1.25 g of DCA and 2.5 g of D-limonene were combined in 25 ml aqueous solution, and distributed into 10 ml aliquots. 0.09 g of aggregated cholesteryl oleate and 0.11 g cholesterol crystals were placed in independent DCA-D-limonene aliquots, creating test samples. Each test sample was incubated at room temperature for two hours with continuous stirring.

A control aqueous solution was distributed into 10 ml aliquots, and 0.09 g of aggregated cholesteryl oleate and 0.11 g of cholesterol crystals were placed in independent control aliquots, creating control samples. The control samples were processed in the same manner as the test samples: i.e., incubated at room temperature for two hours with continuous stirring.

After incubation, 100% of each of the aggregated cholesteryl oleate and cholesterol crystals of the test samples had dissolved. In contrast, the aggregated cholesteryl oleate, cholesteryl palmitate, and cholesterol crystals of the control samples remained insoluble.

These experiments demonstrate that combinations of DCA and D-limonene have a property of being a significantly effective emulsifier of aggregated atherosclerotic plaque lipids. It is believed that these results indicate a substantial number of bile acid and terpene combinations are effective emulsifiers of atherosclerotic plaques in vivo.

### Experiment 6

Ex vivo experiments were performed to assess the ability of DCA to solubilize atherosclerotic plaque material. In these experiments, ex vivo samples of pig artery were bathed in an aqueous solution at two different concentrations of DCA. In the first experiment, samples were treated with 50 mg/mL DCA for successive periods of 30 minutes, at which time the sample was removed from the bathing medium, and the appearance of the plaque examined macroscopically. Early in the treatment, on removal of the sample from the bath a clear, viscous, column of fluid extended from the sample. This column of fluid continued to be apparent when samples were evaluated up to about 4 or 5 hours, after which the fluid column was no longer noted. Without wishing to be held to any one theory of operation, it was concluded that the clear fluid comprised components of the plaque.

After 5 hours of treatment with DCA, macroscopic assessment of plaque size suggested that plaque volume had decreased by about 70%. After 36 hours of exposure all that appeared to remain of plaques were the fibrous cap material and areas of calcification. All core material appeared to have been solubilized.

In a second experiment, atherosclerotic plaque in a sample of pig artery was exposed to a continuous flow of a solution of 0.25 mg/mL DCA, diluted in normal saline (approximately 600 μM DCA). The sample was continuously exposed for a period of 8 days. Macroscopic examination of the sample at this time revealed that most, if not all, of the lipid core of the plaque had been solubilized, and all that remained was the fibrous cap.

In both experiments, treatment with DCA caused no obvious detrimental effects on the vessel itself. In particular, elasticity of the vessel wall appeared unaffected. While not wishing to be held to any one theory of operation, sustained levels of an emulsifier are demonstrated by this example to be effective to produce regression of atherosclerotic plaque, apparently by dissolving the lipid components of the plaque, which once solubilized cross the fibrous cap into the surrounding milieu. In a patient, it is expected that solubilized lipid liberated from plaques by the administered emulsifiers, will be released into the blood stream where they can be metabolized and eliminated from the body by normal physiological routes, for example, by excretion in the bile as free cholesterol, or by conversion to bile acids in the liver.

The skilled artisan will recognize the interchangeability of various features from different embodiments. Similarly, the various features and steps discussed above, as well as other known equivalents for each such feature or step, can be mixed and matched by one of ordinary skill in this art to perform compositions or methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

What is claimed is:

1. A method of treating atherosclerosis in a patient in need thereof, comprising: administering to said patient a pharmaceutical formulation comprising a therapeutically effective amount of ursodeoxycholic acid (UDCA), or a pharmaceutically acceptable salt, hydrate, solvate, or polymorph thereof, at a dose from greater than 15 mg/kg/day to about 100 mg/kg/day.

2. The method of claim 1, wherein said UDCA, or a pharmaceutically acceptable salt, hydrate, solvate, or polymorph thereof, is administered at a dose from greater than 15 mg/kg/day to about 20 mg/kg/day.

3. The method of claim 1, wherein said UDCA, or a pharmaceutically acceptable salt, hydrate, solvate, or polymorph thereof, is administered at a dose from about 20 mg/kg/day to about 50 mg/kg/day.

4. The method of claim 1, wherein said UDCA, or a pharmaceutically acceptable salt, hydrate, solvate, or polymorph thereof, is administered at a dose from about 20 mg/kg/day to about 30 mg/kg/day.

5. The method of claim 1, wherein said UDCA, or a pharmaceutically acceptable salt, hydrate, solvate, or polymorph thereof, is administered in an amount to achieve a concentration of from about 10 μM to about 20 μM in the patient's systemic circulation.

6. The method of claim 1, wherein said UDCA, or a pharmaceutically acceptable salt, hydrate, solvate, or polymorph thereof, is administered in an amount to achieve a concentration of from about 20 μM to about 30 μM in the patient's systemic circulation.

7. The method of claim 1, wherein said administration results in a sustained level of UDCA in the patient's systemic circulation for a period of at least two hours.

8. The method of claim 6, wherein said sustained level of UDCA is achieved within about five minutes after onset of administration.

9. The method of claim 1, wherein the pharmaceutical formulation further comprises a statin.

10. The method of claim 1, further comprising administering a statin with said pharmaceutical formulation.

11. The method of claim 9, wherein said statin is administered at a dose from about 1 mg/day to about 10 mg/day.

12. The method of claim 9, wherein said statin is administered at a dose from about 10 mg/day to about 20 mg/day.

13. The method of claim 9, wherein said statin is administered at a dose from about 20 mg/day to about 30 mg/day.

14. The method of claim 9, wherein said statin is atorvastatin, cerivastatin, fluvastatin, lovastatin, mevastatin, pitavastatin, pravastatin, rosuvastatin, or simvastatin.

15. The method of claim 9, wherein said statin is atorvastatin.

16. The method of claim 1, further comprising administering a second bile acid to said patient.

17. The method of claim 15, wherein said second bile acid is: cholic acid, chenodeoxycholic acid, deoxycholic acid, glycocholic acid, taurocholic acid, hyodeoxycholic acid, lithocholic acid, or a pharmaceutically acceptable salt, hydrate, solvate, or polymorph thereof.

18. The method of claim 16, wherein said UDCA and second bile acid are administered in an amount to achieve a concentration of greater than 50 μM of total bile acids in the patient's systemic circulation.

19. The method of claim 1, wherein said formulation further comprises a saponin, a detergent, or a mixture thereof.

20. The method of claim 1, wherein said formulation further comprises a permeability enhancer.
